(19) United States
(12) Patent Application Publication
(10) Pub. No.: US 2010/0258118 A1
(43) Pub. Date: Oct. 14, 2010

(54) INHALER DEVICES AND BESPOKE PHARMACEUTICAL COMPOSITIONS

(75) Inventor: David Morton, Wiltshire (GB)

Correspondence Address: Davidson, Davidson & Kappel, LLC, 485 7th Avenue, 14th Floor, New York, NY, 10018, US

(73) Assignee: Vectura Limited, Chippenham (GB)

(21) Appl. No.: 12/312,270

(22) PCT Filed: Nov. 5, 2007
(86) PCT No.: PCT/GB2007/050674

§ 371 Date: Jun. 29, 2010

(30) Foreign Application Priority Data
Nov. 3, 2006 (GB) 0621957.0

(51) Int. Cl. A61M 15/00; B29B 9/00

(52) U.S. Cl. 128/203.15; 264/13; 264/10; 264/9

(57) ABSTRACT

The present invention relates to inhaler devices and bespoke pharmaceutical dry powder composition to be dispensed using such inhaler devices for pulmonary administration. In particular, the present invention relates to the provision of passive inhaler devices and dry powder compositions which are specifically formulated and prepared to be efficiently dispensed by such devices to reproducibly achieve a high delivered dose of the pharmaceutically active agent.

The present invention relates to inhaler devices and bespoke pharmaceutical dry powder composition to be dispensed using such inhaler devices for pulmonary administration. In particular, the present invention relates to the provision of passive inhaler devices and dry powder compositions which are specifically formulated and prepared to be efficiently dispensed by such devices to reproducibly achieve a high delivered dose of the pharmaceutically active agent.

The present invention is concerned with the optimisation of the combination of passive dry powder inhaler device and dry powder composition.

Dry powder inhalers (DPIs) are well known in the art and there are a variety of different types. Generally, the dry powder is stored within the device and is extracted from the place of storage upon actuation of the device, whereupon the powder is expelled from the device in the form of a plume of powder which is to be inhaled by the subject. In most DPIs, the powder is stored in a unitary manner, for example in blisters or capsules containing a predetermined amount of the dry powder formulation. Some DPIs have a powder reservoir and doses of the powder are measured out within the device. These reservoir devices are less favoured in the present invention as the blisters or capsules tend to provide more accurate doses.

So-called “passive” DPIs are those in which the patient's breath is the only source of gas which provides a motive force in the device. Examples of “passive” dry powder inhaler devices include Rotahaler™ and Diskhaler™ (GlaxoSmithKline), Turbohaler™ (Astra-Draco), Novolizer™ (Viatris GmbH), Monohaler™ (Miat) and Gyrohaler™ (Vectura). “Active” DPIs are those in which a source of compressed gas or alternative energy source is used. Examples of suitable active devices include Aspirair™ (Vectura), the Microdose™ device and the active inhaler device produced by Nektar Therapeutics.

Conventionally, whilst passive devices are frequently simpler and cheaper, they tend to be less efficient at delivering the active agent in the dry powder composition to the deep lung than active devices. This is because it is more difficult to entrain the powder held in a blister or capsule using the patient's breath than it is to entrain it in a gas flow generated by the device. The patient's breath is more unpredictable and often less powerful than the gas flow generated by active devices. The gas flow is important because it entrains the powder stored within the blister or capsule inside the device. The gas flow needs to create sufficient turbulence to separate powder particles and to pick them up and carry them out of the device. The gas flow should also scour the blister or capsule wall to dislodge any particles adhered thereto, thereby ensuring that as much of the metered dose as possible is dispensed. The gas flow exits the device as a cloud of powder particles in which the fine active particles should be present in a largely deagglomerated form, so that they have a MMAD suitable to allow inhalation and deep lung deposition. Finally, the particles need to travel at a velocity within the cloud or plume that minimises deposition of active particles in the patient's mouth and throat and maximises deposition in the lung.

In light of the foregoing, dry powder delivery systems where a high dosing efficiency is required will usually comprise an active DPI. However, the present invention is concerned with high efficiency drug delivery systems and/or systems exhibiting high reproducibility, the systems comprising dry powder formulations dispensed using passive DPIs.

High dosing efficiency will have a variety of benefits. For example, as it is possible to repeatedly and reliably deliver a higher proportion of the active agent in a dose, it will be possible to reduce the size of the doses whilst still achieving the same therapeutic effect.

The systems disclosed herein provide high dose reproducibility. The reproducibility is measured in terms of relative standard deviation (RSD %) and is in the order of less than 10, less than 7.5, less than 5, less than 4 or less than 3%. Additionally, the lower dose and the high reproducibility achieved by the present invention mean that the therapeutic effect achieved by a given dose will be more predictable and consistent. This obviates the risk of having an unexpected and unusually high dosing efficiency with the conventional devices and powders, which could lead to an undesirably high dose of active agent being administered, effectively an overdose.

Furthermore, high doses of therapeutically active agents have long been linked with the increased incidence of undesirable side effects. Thus, the present invention may help to reduce the incidence of side effects by reducing the dose administered to all patients.

Yet another advantage associated with the higher dosing efficiency of the present invention is that it may be possible to achieve a longer-lasting therapeutic effect without having to increase the dose administered to the patient. The greater dosing efficiency means that a greater amount of a given dose is actually delivered. This can lead to a greater therapeutic effect and, in cases where the active agent does not have a short half-life, this will also mean that the therapeutic effect lasts for a longer period of time. In some circumstances, this may even mean that it is possible to use the present invention to administer an active agent in an immediate release form and achieve the same extended therapeutic effect as a sustained release form of the same active agent.

Naturally, the reduction in the amount of an active agent required to achieve the same therapeutic effect is attractive because of the cost implications. However, it is also likely to be deemed much safer by regulatory bodies such as the FDA in the United States.

Yet another advantage associated with the reduced throat deposition, in that any unpleasant taste effects of the active will be minimised. Also, any side effects such as throat infections caused by deposition of steroids on the throat are reduced.

A particular advantage which is afforded by the high dosing efficiency achieved by the present invention is that it confirms that administration of pharmaceutically active agents in the form of a dry powder and via pulmonary inhalation is an effective and efficient mode of administration. The serum concentration of the active agent following the administration of a dry powder formulation by pulmonary inhalation according to the present invention has been shown to be consistent between doses and between different individuals. There is no variation between individuals, as is observed with other modes of administration (such as oral administration). This means that the therapeutic effect of the administration of a given dose is predictable and reliable. This has the added benefit that a balance can more easily be struck between the therapeutic effect of a pharmaceutically active agent and any adverse effects that might be associated with its administration.

The reason for the lack of dosing efficiency seen in many conventional dry powder delivery systems is that a proportion of the active agent in the dose of dry powder tends to be effectively lost at every stage the powder goes through; substantial amounts of the active agent may remain in the device and not all of the active agent that makes it out of the device will be inhaled and deposited in the lung, as some of the active material may be deposited in the throat of the subject due to excessive plume velocity. Further, poor matching of the device and powder formulation can result in variability and inconsistency in dosing. To date, little has been done to match passive DPIs and dry powder compositions in order to optimise the pulmonary delivery of the active agent.

The metered dose (MD) of a dry powder formulation is the total mass of active agent present in the metered form presented by the inhaler device in question. For example, the MD might be the mass of active agent present in a capsule or in a foil blister.

The emitted dose (ED) is the total mass of the active agent emitted from the device following actuation. It does not include the material left inside or on the surfaces of the device. The ED is measured by collecting the total emitted mass from the device in an apparatus frequently identified as a dose uniformity sampling apparatus (DUSA), and recovering this by a validated quantitative wet chemical assay.

The fine particle dose (FPD) is the total mass of active agent which is emitted from the device following actuation which is present in an aerodynamic particle size smaller than a defined limit. This limit is generally taken to be 5 μm if not expressly stated to be an alternative limit, such as 3 μm or 1 μm, etc. The FPD is measured using an impactor or impinger, such as a twin stage impinger (TSI), multi-stage impinger (MSI), Andersen Cascade Impactor or a Next Generation Impactor (NGI). Each impactor or impinger has a pre-determined aerodynamic particle size collection cut points for each stage. The FPD value is obtained by interpretation of the stage-by-stage active agent recovery quantified by a validated quantitative wet chemical assay where either a simple stage cut is used to determine FPD or a more complex mathematical interpolation of the stage-by-stage deposition is used.

The fine particle fraction (FPF) is normally defined as the FPD divided by the ED and expressed as a percentage. Herein, the FPF of ED is referred to as FPF(ED) and is calculated as FPF(ED)=(FPD/ED)×100%.

The fine particle fraction (FPF) may also be defined as the FPD divided by the MD and expressed as a percentage. Herein, the FPF of MD is referred to as FPF(MD), and is calculated as FPF(MD)=(FPD/MD)×100%.

The FPF(MD) can also be termed the ‘Dose Efficiency’ and is the amount of the dose of the pharmaceutical dry powder formulation which, upon being dispensed from the delivery device, is below a specified aerodynamic particle size.

Whilst the FPF and FPD of a dry powder formulation are dependent on the nature of the powder itself, these values are clearly also influenced by the type of inhaler used to dispense the powder. As a rule, the FPF observed when dispensing a dry powder composition using a passive device will not to be as good as that observed when the same powder is dispensed using an active device, such as an Aspirair (trade mark) device (see WO 01/00262 and GB2353222).

It is an aim of the present invention to provide a drug delivery system which provides improved FPF and FPD values upon dispensing the dry powder formulation using a passive device, so that the FPF and FPD are at least as high and/or as consistent as those observed with active device delivery, and preferably better.

It is a particular aim of the present invention to provide a drug delivery system which provides an FPF of at least 35%. Preferably, the FPF(ED) will be between 40 and 99%, between 50 and 99%, between 60 and 99%, between 70 and 99%, or between 80 and 99%. Furthermore, it is desirable for the FPF(MD) to be at least 30%. Preferably, the FPF(MD) will be between 40 and 99%, between 50 and 99%, or between 60 and 99%.

Thus, according to a first aspect of the present invention, there is provided a drug delivery system comprising a passive dry powder inhaler device and a dry powder composition, wherein the powder composition comprises a pharmaceutically active agent and wherein the combination of the device and the composition ensure that at least 50% of the metered dose of the active agent is deposited in the lung. Preferably, at least 60% of the metered dose of the active agent is deposited in the lung.

In a preferred embodiment of the present invention, the amount of active agent retained in the blister or capsule following actuation of the device is less than 15%, preferably less than 10%, more preferably less than 7% and most preferably less than 5% or 3%.

In another preferred embodiment, the amount of the powder formulation retained in the dispensing device, for example in the blister or capsule, in the mouthpiece and in any other device part, is less than 15%, preferably less than 10%, more preferably less than 7% and most preferably less than 5% or 3%.

In a yet further embodiment, upon being expelled from the dispensing device, the powder formulation has a dosing efficiency at 5 μm of preferably at least 40%, at least 50%, at least 60%, at least 70%, at least 75%, at least 80%, at least 85%, at least 90%, or at least 95%.

Preferably, upon being expelled from the dispensing device, the powder formulation has a dosing efficiency at 3 μm of preferably at least 30%, at least 40%, at least 50%, at least 60%, at least 70%, at least 75%, at least 80%, at least 85%, or at least 90%.

Preferably, upon being expelled from the dispensing device, the powder formulation has a dosing efficiency at 2 μm of preferably at least 20%, at least 30%, at least 40%, at least 50%, at least 55%, at least 60%, or at least 70%. These efficiencies are far greater than anything consistently achieved prior to this invention using a passive dry powder inhaler device.

In another preferred embodiment, the particles comprising a pharmaceutically active agent (active particles) have a mass median aerodynamic diameter (MMAD) of less than 10 μm. Preferably the MMAD of the active particles is less than 7 μm, more preferably less than 5 μm, more preferably less than 2 μm, and most preferably less than 1.5 μm.

Finally, in another preferred embodiment, the amount of the active agent which is deposited in the throat of the user is less than 15% of the active agent in the metered dose. Preferably, throat deposition is less than 10%, more preferably it is less than 7% and most preferably it is less than 5% or less than 3%.

High dosing efficiency requires the balancing of various factors which affect the extraction of the powder formulation from the dispensing device, the dynamics of the powder plume created by the device and the deposition of the active particles within the lung. One of the factors affecting these is the tendency of the powder particles to agglomerate. This, in turn, is linked to the size of the particles, as well as other factors, such as the presence of force controlling agents on the surface of the powder particles, particle morphology and density, as well as the type of device used to dispense the powder.

It is essential for the powder properties to be appropriately balanced for passive device delivery. Fine particles which do not agglomerate will, on the one hand, be beneficial as all of the particles will be of the appropriate size for lung deposition. However, powder formulations comprising such non-agglomerating particles will tend to have poor flow characteristics, which will make extraction of the powder from the inhaler device difficult, potentially leading to loss of dosing efficiency as a result of increased device retention. If the flowability of the powder is improved, the extraction of the powder from the device is also likely to be improved. However, if the extraction of the powder becomes too easy, this can also have a detrimental effect, which is probably more marked where an active type of dry powder inhaler device is used. As a result of the improved flowability and easier extraction of the powder, it is possible that the powder will actually leave the device too quickly. This can mean that the active particles travel too quickly within the powder plume generated by the device and these particles therefore tend to impact on the subject's throat rather than being inhaled. Thus, the dosing efficiency is once again reduced, this time as a result of increased throat impaction or deposition.

The present invention can be carried out with any pharmaceutically active agent. Specific active agents or drugs that may be used include, but are not limited to, agents of one or more of the following classes listed below.

1) Adrenergic agonists such as, for example, amphetamine, apraclonidine, bitolterol, clonidine, colterol, dobutamine, dopamine, ephedrine, epinephrine, ethylnorepinephrine, fenoterol, formoterol, guanabenz, guanfacine, hydroxyamphetamine, isoetharine, isoproterenol, isotharine, mephenterine, metaraminol, methamphetamine, methoxamine, methpentermine, methyldopa, methylphenidate, metaproterenol, metaraminol, mitodrine, naphazoline, norepinephrine, oxymetazoline, pemoline, phenylephrine, phenylethylamine, phenylpropanolamine, pirbuterol, prenalterol, procaterol, propylhexedrine, pseudo-ephedrine, ritodrine, salbutamol, salmeterol, terbutaline, tetrahydrozoline, tramazoline, tyramine and xylometazoline.
2) Adrenergic antagonists such as, for example, acebutolol, alfuzosin, atenolol, betaxolol, bisoprolol, bopindolol, bucindolol, bunazosin, butyrophenones, carteolol, carvedilol, celiprolol, chlorpromazine, doxazosin, ergot alkaloids, esmolol, haloperidol, indoramin, ketanserin, labetalol, levobunolol, medroxalol, metipranolol, metoprolol, nebivolol, nadolol, naftopidil, oxprenolol, penbutolol, phenothiazines, phenoxybenzamine, phentolamine, pindolol, prazosin, propafenone, propranolol, sotalol, tamsulosin, terazosin, timolol, tolazoline, trimazosin, urapidil and yohimbine.
3) Adrenergic neurone blockers such as, for example, bethanidine, debrisoquine, guabenxan, guanadrel, guanazodine, guanethidine, guanoclor and guanoxan.
4) Drugs for treatment of addiction, such as, for example, buprenorphine.
5) Drugs for treatment of alcoholism, such as, for example, disulfuram, naloxone and naltrexone.
6) Drugs for Alzheimer's disease management, including acetylcholinesterase inhibitors such as, for example, donepezil, galantamine, rivastigmine and tacrin.
7) Anaesthetics such as, for example amethocaine, benzocaine, bupivacaine, hydrocortisone, ketamine, lignocaine, methylprednisolone, prilocaine, proxymetacaine, ropivacaine and tyrothricin.
8) Angiotensin converting enzyme inhibitors such as, for example, captopril, cilazapril, enalapril, fosinopril, imidapril hydrochloride, lisinopril, moexipril hydrochloride, perindopril, quinapril, ramipril and trandolapril.
9) Angiotensin II receptor blockers, such as, for example, candesartan, cilexetil, eprosartan, irbesartan, losartan, medoxomil, olmesartan, telmisartan and valsartan.
10) Antiarrhythmics such as, for example, adenosine, amidodarone, disopyramide, flecainide acetate, lidocaine hydrochloride, mexiletine, procainamide, propafenone and quinidine.
11) Antibiotic and antibacterial agents (including the beta-lactams, fluoroquinolones, ketolides, macrolides, sulphonamides and tetracyclines) such as, for example, aclarubicin, amoxicillin, amphotericin, azithromycin, aztreonam chlorhexidine, clarithromycin, clindamycin, colistimethate, dactinomycin, dirithromycin, doripenem, erythromycin, fusafungine, gentamycin, metronidazole, mupirocin, natamycin, neomycin, nystatin, oleandomycin, pentamidine, pimaricin, probenecid, roxithromycin, sulphadiazine and triclosan.
12) Anti-clotting agents such as, for example, abciximab, acenocoumarol, alteplase, aspirin, bemiparin, bivalirudin, certoparin, clopidogrel, dalteparin, danaparoid, dipyridamole, enoxaparin, epoprostenol, eptifibatide, fondaparin, heparin (including low molecular weight heparin), heparin calcium, lepirudin, phenindione, reteplase, streptokinase, tenecteplase, tinzaparin, tirofiban and warfarin.
13) Anticonvulsants such as, for example, GABA analogs including tiagabine and vigabatrin; barbiturates including pentobarbital; benzodiazepines including alprazolam, chlordiazepoxide, clobazam, clonazepam, diazepam, flurazepam, lorazepam, midazolam, oxazepam and zolazepam; hydantoins including phenyloin; phenyltriazines including lamotrigine; and miscellaneous anticonvulsants including acetazolamide, carbamazepine, ethosuximide, fosphenytoin, gabapentin, levetiracetam, oxcarbazepine, piracetam, pregabalin, primidone, sodium valproate, topiramate, valproic acid and zonisamide.
14) Antidepressants such as, for example, tricyclic and tetracyclic antidepressants including amineptine, amitriptyline (tricyclic and tetracyclic amitryptiline), amoxapine, butriptyline, cianopramine, clomipramine, demexiptiline, desipramine, dibenzepin, dimetacrine, dosulepin, dothiepin, doxepin, imipramine, iprindole, levoprotiline, lofepramine, maprotiline, melitracen, metapramine, mianserin, mirtazapine, nortryptiline, opipramol, propizepine, protriptyline, quinupramine, setiptiline, tianeptine and trimipramine; selective serotonin and noradrenaline reuptake inhibitors (SNRIs) including clovoxamine, duloxetine, milnacipran and venlafaxine; selective serotonin reuptake inhibitors (SSRIs) including citalopram, escitalopram, femoxetine, fluoxetine, fluvoxamine, ifoxetine, milnacipran, nomifensine, oxaprotiline, paroxetine, sertraline, sibutramine, venlafaxine, viqualine and zimeldine; selective noradrenaline reuptake inhibitors (NARIs) including demexiptiline, desipramine, oxaprotiline and reboxetine; noradrenaline and selective serotonin reuptake inhibitors (NASSAs) including mirtazapine; monoamine oxidase inhibitors (MAOIs) including amiflamine, brofaromine, clorgyline, α-ethyltryptamine, etoperidone, iproclozide, iproniazid, isocarboxazid, mebanazine, medifoxamine, moclobemide, nialamide, pargyline, phenelzine, pheniprazine, pirlindole, procarbazine, rasagiline, safrazine, selegiline, toloxatone and tranylcypromine; muscarinic antagonists including benactyzine and dibenzepin; azaspirones including buspirone, gepirone, ipsapirone, tandospirone and tiaspirone; and other antidepressants including acetaphenazine, ademetionine, S-adenosylmethionine, adrafinil, amesergide, amineptine, amperozide, benactyzine, benmoxine, binedaline, bupropion, carbamazepine, caroxazone, cericlamine, cotinine, fezolamine, flupentixol, idazoxan, kitanserin, levoprotiline, lithium salts, maprotiline, medifoxamine, methylphenidate, metralindole, minaprine, nefazodone, nisoxetine, nomifensine, oxaflozane, oxitriptan, phenyhydrazine, rolipram, roxindole, sibutramine, teniloxazine, tianeptine, tofenacin, trazadone, tryptophan, viloxazine and zalospirone.
15) Anticholinergic agents such as, for example, atropine, benzatropine, biperiden, cyclopentolate, glycopyrrolate, hyoscine, ipratropium bromide, orphenadine hydrochloride, oxitroprium bromide, oxybutinin, pirenzepine, procyclidine, propantheline, propiverine, telenzepine, tiotropium, trihexyphenidyl, tropicamide and trospium.
16) Antidiabetic agents such as, for example, pioglitazone, rosiglitazone and troglitazone.
17) Antidotes such as, for example, deferoxamine, edrophonium chloride, fiumazenil, nalmefene, naloxone, and naltrexone.
18) Anti-emetics such as, for example, alizapride, azasetron, benzquinamide, bestahistine, bromopride, buclizine, chlorpromazine, cinnarizine, clebopride, cyclizine, dimenhydrinate, diphenhydramine, diphenidol, domperidone, dolasetron, dronabinol, droperidol, granisetron, hyoscine, lorazepam, metoclopramide, metopimazine, nabilone, ondansetron, palonosetron, perphenazine, prochlorperazine, promethazine, scopolamine, triethylperazine, trifluoperazine, triflupromazine, trimethobenzamide and tropisetron.
19) Antihistamines such as, for example, acrivastine, astemizole, azatadine, azelastine, brompheniramine, carbinoxamine, cetirizine, chlorpheniramine, cinnarizine, clemastine, cyclizine, cyproheptadine, desloratadine, dexmedetomidine, diphenhydramine, doxylamine, fexofenadine, hydroxyzine, ketotifen, levocabastine, loratadine, mizolastine, promethazine, pyrilamine, terfenadine and trimeprazine.
20) Anti-infective agents such as, for example, antivirals (including nucleoside and non-nucleoside reverse transcriptase inhibitors and protease inhibitors) including aciclovir, adefovir, amantadine, cidofovir, efavirenz, famiciclovir, foscarnet, ganciclovir, idoxuridine, indinavir, inosine pranobex, lamivudine, nelfinavir, nevirapine, oseltamivir, palivizumab, penciclovir, pleconaril, ribavirin, rimantadine, ritonavir, ruprintrivir, saquinavir, stavudine, valaciclovir, zalcitabine, zanamivir, zidovudine and interferons; AIDS adjunct agents including dapsone; aminoglycosides including tobramycin; antifungals including amphotericin, caspofungin, clotrimazole, econazole nitrate, fluconazole, itraconazole, ketoconazole, miconazole, nystatin, terbinafine and voriconazole; anti-malarial agents including quinine; antituberculosis agents including capreomycin, ciprofloxacin, ethambutol, meropenem, piperacillin, rifampicin and vancomycin; beta-lactams including cefazolin, cefmetazole, cefoperazone, cefoxitin, cephacetrile, cephalexin, cephaloglycin and cephaloridine; cephalosporins, including cephalosporin C and cephalothin; cephamycins such as cephamycin A, cephamycin B, cephamycin C, cephapirin and cephradine; leprostatics such as clofazimine; penicillins including amoxicillin, ampicillin, amylpenicillin, azidocillin, benzylpenicillin, carbenicillin, carfecillin, carindacillin, clometocillin, cloxacillin, cyclacillin, dicloxacillin, diphenicillin, heptylpenicillin, hetacillin, metampicillin, methicillin, nafcillin, 2-pentenylpenicillin, penicillin N, penicillin O, penicillin S and penicillin V; quinolones including ciprofloxacin, clinafloxacin, difloxacin, grepafloxacin, norfloxacin, ofloxacine and temafloxacin; tetracyclines including doxycycline and oxytetracycline; miscellaneous anti-infectives including linezolide, trimethoprim and sulfamethoxazole.
21) Anti-neoplastic agents such as, for example, droloxifene, tamoxifen and toremifene.
22) Antiparkisonian drugs such as, for example, amantadine, andropinirole, apomorphine, baclofen, benserazide, biperiden, benztropine, bromocriptine, budipine, cabergoline, carbidopa, eliprodil, entacapone, eptastigmine, ergoline, galanthamine, lazabemide, levodopa, lisuride, mazindol, memantine, mofegiline, orphenadrine, trihexyphenidyl, pergolide, piribedil, pramipexole, procyclidine, propentofylline, rasagiline, remacemide, ropinerole, selegiline, spheramine, terguride and tolcapone.
23) Antipsychotics such as, for example, acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, loxapine, melperone, mesoridazine, metofenazate, molindrone, olanzapine, penfluridol, pericyazine, perphenazine, pimozide, pipamerone, piperacetazine, pipotiazine, prochlorperazine, promazine, quetiapine, remoxipride, risperidone, sertindole, spiperone, sulpiride, thioridazine, thiothixene, trifluperidol, triflupromazine, trifluoperazine, ziprasidone, zotepine and zuclopenthixol; phenothiazines including aliphatic compounds, piperidines and piperazines; thioxanthenes, butyrophenones and substituted benzamides.
24) Antirheumatic agents such as, for example, diclofenac, heparinoid, hydroxychloroquine and methotrexate, leflunomide and teriflunomide.
25) Anxiolytics such as, for example, adinazolam, alpidem, alprazolam, alseroxlon, amphenidone, azacyclonol, bromazepam, bromisovalum, buspirone, captodiamine, capuride, carbcloral, carbromal, chloral betaine, chlordiazepoxide, clobenzepam, enciprazine, flesinoxan, flurazepam, hydroxyzine, ipsapiraone, lesopitron, loprazolam, lorazepam, loxapine, mecloqualone, medetomidine, methaqualone, methprylon, metomidate, midazolam, oxazepam, propanolol, tandospirone, trazadone, zolpidem and zopiclone.
26) Appetite stimulants such as, for example, dronabinol.
27) Appetite suppressants such as, for example, fenfluramine, phentermine and sibutramine; and anti-obesity treatments such as, for example, pancreatic lipase inhibitors, serotonin and norepinephrine re-uptake inhibitors, and anti-anorectic agents.
28) Benzodiazepines such as, for example, alprazolam, bromazepam, brotizolam, chlordiazepoxide, clobazam, clonazepam, clorazepate, demoxepam, diazepam, estazolam, flunitrazepam, flurazepam, halazepam, ketazolam, loprazolam, lorazepam, lormetazepam, medazepam, midazolam, nitrazepam, nordazepam, oxazepam, prazepam, quazepam, temazepam and triazolam.
29) Bisphosphonates such as, for example, alendronate sodium, sodium clodronate, etidronate disodium, ibandronic acid, pamidronate disodium, isedronate sodium, tiludronic acid and zoledronic acid.
30) Blood modifiers such as, for example, cilostazol and dipyridamol, and blood factors.
31) Cardiovascular agents such as, for example, acebutalol, adenosine, amiloride, amiodarone, atenolol, benazepril, bisoprolol, bumetanide, candesartan, captopril, clonidine, diltiazem, disopyramide, dofetilide, doxazosin, enalapril, esmolol, ethacrynic acid, flecanide, furosemide, gemfibrozil, ibutilide, irbesartan, labetolol, losartan, lovastatin, metolazone, metoprolol, mexiletine, nadolol, nifedipine, pindolol, prazosin, procainamide, propafenone, propranolol, quinapril, quinidine, ramipril, sotalol, spironolactone, telmisartan, tocainide, torsemide, triamterene, valsartan and verapamil.
32) Calcium channel blockers such as, for example, amlodipine, bepridil, diltiazem, felodipine, flunarizine, gallopamil, isradipine, lacidipine, lercanidipine, nicardipine, nifedipine, nimodipine and verapamil.
33) Central nervous system stimulants such as, for example, amphetamine, brucine, caffeine, dexfenfluramine, dextroamphetamine, ephedrine, fenfluramine, mazindol, methyphenidate, modafmil, pemoline, phentermine and sibutramine.
34) Cholesterol-lowering drugs such as, for example, acipimox, atorvastatin, ciprofibrate, colestipol, colestyramine, bezafibrate, ezetimibe, fenofibrate, fluvastatin, gemfibrozil, ispaghula, nictotinic acid, omega-3 triglycerides, pravastatin, rosuvastatin and simvastatin.
35) Drugs for cystic fibrosis management such as, for example, Pseudomonas aeruginosa infection vaccines (eg Aerugen™), alpha 1-antitripsin, amikacin, cefadroxil, denufosol, duramycin, glutathione, mannitol, and tobramycin.
36) Diagnostic agents such as, for example, adenosine and aminohippuric acid.
37) Dietary supplements such as, for example, melatonin and vitamins including vitamin E.
38) Diuretics such as, for example, amiloride, bendroflumethiazide, bumetanide, chlortalidone, cyclopenthiazide, furosemide, indapamide, metolazone, spironolactone and torasemide.
39) Dopamine agonists such as, for example, amantadine, apomorphine, bromocriptine, cabergoline, lisuride, pergolide, pramipexole and ropinerole.
40) Drugs for treating erectile dysfunction, such as, for example, apomorphine, apomorphine diacetate, moxisylyte, phentolamine, phosphodiesterase type 5 inhibitors, such as sildenafil, tadalafil, vardenafil and yohimbine.
41) Gastrointestinal agents such as, for example, atropine, hyoscyamine, famotidine, lansoprazole, loperamide, omeprazole and rebeprazole.
42) Hormones and analogues such as, for example, cortisone, epinephrine, estradiol, insulin, Ostabolin-C, parathyroid hormone and testosterone.
43) Hormonal drugs such as, for example, desmopressin, lanreotide, leuprolide, octreotide, pegvisomant, protirelin, salcotonin, somatropin, tetracosactide, thyroxine and vasopressin.
44) Hypoglycaemics such as, for example, sulphonylureas including glibenclamide, gliclazide, glimepiride, glipizide and gliquidone; biguanides including metformin; thiazolidinediones including pioglitazone, rosiglitazone, nateglinide, repaglinide and acarbose.

### 45) Immunoglobulins.

46) Immunomodulators such as, for example, interferon (e.g. interferon beta-1a and interferon beta-1b) and glatiramer.
47) Immunosupressives such as, for example, azathioprine, cyclosporin, mycophenolic acid, rapamycin, sirolimus and tacrolimus.
48) Mast cell stabilizers such as, for example, cromoglycate, iodoxamide, nedocromil, ketotifen, tryptase inhibitors and pemirolast.
49) Drugs for treatment of migraine headaches such as, for example, almotriptan, alperopride, amitriptyline, amoxapine, atenolol, clonidine, codeine, coproxamol, cyproheptadine, dextropropoxypene, dihydroergotamine, diltiazem, doxepin, ergotamine, eletriptan, fluoxetine, frovatriptan, isometheptene, lidocaine, lisinopril, lisuride, loxapine, methysergide, metoclopramide, metoprolol, nadolol, naratriptan, nortriptyline, oxycodone, paroxetine, pizotifen, pizotyline, prochlorperazine propanolol, propoxyphene, protriptyline, rizatriptan, sertraline, sumatriptan, timolol, tolfenamic acid, tramadol, verapamil, zolmitriptan, and non-steroidal anti-inflammatory drugs.
50) Drugs for treatment of motion sickness such as, for example, diphenhydramine, promethazine and scopolamine.
51) Mucolytic agents such as N-acetylcysteine, ambroxol, amiloride, dextrans, heparin, desulphated heparin, low molecular weight heparin and recombinant human DNase.
52) Drugs for multiple sclerosis management such as, for example, bencyclane, methylprednisolone, mitoxantrone and prednisolone.
53) Muscle relaxants such as, for example, baclofen, chlorzoxazone, cyclobenzaprine, methocarbamol, orphenadrine, quinine and tizanidine.
54) NMDA receptor antagonists such as, for example, mementine.
55) Nonsteroidal anti-inflammatory agents such as, for example, aceclofenac, acetaminophen, alminoprofen, amfenac, aminopropylon, amixetrine, aspirin, benoxaprofen, bromfenac, bufexamac, carprofen, celecoxib, choline, cinchophen, cinmetacin, clometacin, clopriac, diclofenac, diclofenac sodium, diflunisal, ethenzamide, etodolac, etoricoxib, fenoprofen, flurbiprofen, ibuprofen, indomethacin, indoprofen, ketoprofen, ketorolac, loxoprofen, mazipredone, meclofenamate, mefenamic acid, meloxicam, nabumetone, naproxen, nimesulide, parecoxib, phenylbutazone, piroxicam, pirprofen, rofecoxib, salicylate, sulindac, tiaprofenic acid, tolfenamate, tolmetin and valdecoxib.
56) Nucleic-acid medicines such as, for example, oligonucleotides, decoy nucleotides, antisense nucleotides and other gene-based medicine molecules.
57) Opiates and opioids such as, for example, alfentanil, allylprodine, alphaprodine, anileridine, benzylmorphine, bezitramide, buprenorphine, butorphanol, carbiphene, cipramadol, clonitazene, codeine, codeine phosphate, dextromoramide, dextropropoxyphene, diamorphine, dihydrocodeine, dihydromorphine, diphenoxylate, dipipanone, fentanyl, hydromorphone, L-alpha acetyl methadol, levorphanol, lofentanil, loperamide, meperidine, meptazinol, methadone, metopon, morphine, nalbuphine, nalorphine, oxycodone, papavereturn, pentazocine, pethidine, phenazocine, pholcodeine, remifentanil, sufentanil, tramadol, and combinations thereof with an anti-emetic.
58) Opthalmic preparations such as, for example, betaxolol and ketotifen.
59) Osteoporosis preparations such as, for example, alendronate, estradiol, estropitate, raloxifene and risedronate.
60) Other analgesics such as, for example, apazone, benzpiperylon, benzydamine, caffeine, cannabinoids, clonixin, ethoheptazine, flupirtine, nefopam, orphenadrine, pentazocine, propacetamol and propoxyphene.
61) Other anti-inflammatory agents such as, for example, B-cell inhibitors, p38 MAP kinase inhibitors and TNF inhibitors.
62) Phosphodiesterase inhibitors such as, for example, non-specific phosphodiesterase inhibitors including theophylline, theobromine, IBMX, pentoxifylline and papaverine; phosphodiesterase type 3 inhibitors including bipyridines such as milrinone, amrinone and olprinone; imidazolones such as piroximone and enoximone; imidazolines such as imazodan and 5-methyl-imazodan; imidazo-quinoxalines; and dihydropyridazinones such as indolidan and LY181512 (5-(6-oxo-1,4,5,6-tetrahydro-pyridazin-3-yl)-1,3-dihydro-indol-2-one); dihydroquinolinone compounds such as cilostamide, cilostazol, and vesnarinone; motapizone; phosphodiesterase type 4 inhibitors such as cilomilast, etazolate, rolipram, oglemilast, roflumilast, ONO 6126, tolafentrine and zardaverine, and including quinazolinediones such as nitraquazone and nitraquazone analogs; xanthine derivatives such as denbufylline and arofylline; tetrahydropyrimidones such as atizoram; and oxime carbamates such as filaminast; and phosphodiesterase type 5 inhibitors including sildenafil, zaprinast, vardenafil, tadalafil, dipyridamole, and the compounds described in WO 01/19802, particularly (S)-2-(2-hydroxymethyl-1-pyrrolidinyl)-4-(3-chloro-4-methoxy-benzylamino)-5-[N-(2-pyrimidinylmethyl)carbamoyl]pyrimidine, 2-(5,6,7,8-tetrahydro-1,7-naphthyridin-7-yl)-4-(3-chloro-4-methoxybenzylamino)-5-[N-(2-morpholinoethyl)carbamoyl]-pyrimidine, and (S)-2-(2-hydroxymethyl-1-pyrrolidinyl)-4-(3-chloro-4-methoxy-benzylamino)-5-[N-(1,3,5-trimethyl-4-pyrazolyl)carbamoyl]-pyrimidine).
63) Potassium channel modulators such as, for example, cromakalim, diazoxide, glibenclamide, levcromakalim, minoxidil, nicorandil and pinacidil.
64) Prostaglandins such as, for example, alprostadil, dinoprostone, epoprostanol and misoprostol.
65) Respiratory agents and agents for the treatment of respiratory diseases including bronchodilators such as, for example, the β₂-agonists bambuterol, bitolterol, broxaterol, carmoterol, clenbuterol, fenoterol, formoterol, indacaterol, levalbuterol, metaproterenol, orciprenaline, picumeterol, pirbuterol, procaterol, reproterol, rimiterol, salbutamol, salmeterol, terbutaline and the like; inducible nitric oxide synthase (iNOS) inhibitors; the antimuscarinics ipratropium, ipratropium bromide, oxitropium, tiotropium, glycopyrrolate and the like; the xanthines aminophylline, theophylline and the like; adenosine receptor antagonists, cytokines such as, for example, interleukins and interferons; cytokine antagonists and chemokine antagonists including cytokine synthesis inhibitors, endothelin receptor antagonists, elastase inhibitors, integrin inhibitors, leukotrine receptor antagonists, prostacyclin analogues, and ablukast, ephedrine, epinephrine, fenleuton, iloprost, iralukast, isoetharine, isoproterenol, montelukast, ontazolast, pranlukast, pseudoephedrine, sibenadet, tepoxalin, verlukast, zafirlukast and zileuton.
66) Sedatives and hypnotics such as, for example, alprazolam, butalbital, chlordiazepoxide, diazepam, estazolam, flunitrazepam, flurazepam, lorazepam, midazolam, temazepam, triazolam, zaleplon, zolpidem, and zopiclone.
67) Serotonin agonists such as, for example, 1-(4-bromo-2,5-dimethoxyphenyl)-2-aminopropane, buspirone, m-chlorophenylpiperazine, cisapride, ergot alkaloids, gepirone, 8-hydroxy-(2-N,N-dipropylamino)-tetraline, ipsaperone, lysergic acid diethylamide, 2-methyl serotonin, mezacopride, sumatriptan, tiaspirone, trazodone and zacopride.
68) Serotonin antagonists such as, for example, amitryptiline, azatadine, chlorpromazine, clozapine, cyproheptadine, dexfenfluramine, R(+)-α-(2,3-dimethoxyphenyl)-1-[2-(4-fluorophenyl)ethyl]-4-piperidine-methanol, dolasetron, fenclonine, fenfluramine, granisetron, ketanserin, methysergide, metoclopramide, mianserin, ondansetron, risperidone, ritanserin, trimethobenzamide and tropisetron.
69) Steroid drugs such as, for example, alcometasone, beclomethasone, beclomethasone dipropionate, betamethasone, budesonide, butixocort, ciclesonide, clobetasol, deflazacort, diflucortolone, desoxymethasone, dexamethasone, fludrocortisone, flunisolide, fluocinolone, fluometholone, fluticasone, fluticasone proprionate, hydrocortisone, methylprednisolone, mometasone, nandrolone decanoate, neomycin sulphate, prednisolone, rimexolone, rofleponide, triamcinolone and triamcinolone acetonide.
70) Sympathomimetic drugs such as, for example, adrenaline, dexamfetamine, dipirefin, dobutamine, dopamine, dopexamine, isoprenaline, noradrenaline, phenylephrine, pseudoephedrine, tramazoline and xylometazoline.
71) Nitrates such as, for example, glyceryl trinitrate, isosorbide dinitrate and isosorbide mononitrate.
72) Skin and mucous membrane agents such as, for example, bergapten, isotretinoin and methoxsalen.
73) Smoking cessation aids such as, for example, bupropion, nicotine and varenicline.
74) Drugs for treatment of Tourette's syndrome such as, for example, pimozide.
75) Drugs for treatment of urinary tract infections such as, for example, darifenicin, oxybutynin, propantheline bromide and tolteridine.

### 76) Vaccines.

77) Drugs for treating vertigo such as, for example, betahistine and meclizine.
78) Therapeutic proteins and peptides such as acylated insulin, glucagon, glucagon-like peptides, exendins, insulin, insulin analogues, insulin aspart, insulin detemir, insulin glargine, insulin glulisine, insulin lispro, insulin zinc, isophane insulins, neutral, regular and insoluble insulins, and protamine zinc insulin.
79) Anticancer agents such as, for example, anthracyclines, doxorubicin, idarubicin, epirubicin, methotrexate, taxanes, paclitaxel, docetaxel, cisplatin, vinca alkaloids, vincristine and 5-fluorouracil.
80) Pharmaceutically acceptable salts or derivatives of any of the foregoing.

It should be noted that drugs listed above under a particular indication or class may also find utility in other indications. A plurality of active agents can be employed in the practice of the present invention. A drug delivery system according to the invention may also be used to deliver combinations of two or more different active agents or drugs. Specific combinations of two medicaments which may be mentioned include combinations of steroids and β₂-agonists. Examples of such combinations are beclomethasone and formoterol; beclomethasone and salmeterol; fluticasone and formoterol; fluticasone and salmeterol; budesonide and formoterol; budesonide and salmeterol; flunisolide and formoterol; flunisolide and salmeterol; ciclesonide and formoterol; ciclesonide and salmeterol; mometasone and formoterol; and mometasone and salmeterol. Specifically drug delivery systems according to the invention may also be used to deliver combinations of three different active agents or drugs.

It will be clear to a person of skill in the art that, where appropriate, the active agents or drugs may be linked to a carrier molecule or molecules and/or used in the form of prodrugs, salts, as esters, or as solvates to optimise the activity and/or stability of the active agent or drug. The device used to deliver the dry powder formulation will clearly affect the performance of the dry powder formulations and the device is therefore a very important part of present invention.

In a preferred embodiment, the passive DPI contains a strip of blisters each having a puncturable lid and containing a dose of the dry powder composition comprising a pharmaceutically active agent for inhalation by a user.

It is common for dry powder formulations to be pre-packaged in individual doses, usually in the form of capsules or blisters which each contain a single dose of the powder which has been accurately and consistently measured. A blister is generally cold formed from a ductile foil laminate or a plastics material and includes a puncturable lid which is permanently heat-sealed around the periphery of the blister during manufacture and after introduction of the dose into the blister. A foil blister is preferred over capsules as each dose is protected from the ingress of water and penetration of gases such as oxygen in addition to being shielded from light and UV radiation all of which can have a detrimental effect on the delivery characteristics of the inhaler if a dose becomes exposed to them. Therefore, a blister offers excellent environmental protection to each individual drug dose.

Inhalation devices which receive a blister pack comprising a number of blisters each of which contain a pre-metered and individually packaged dose of the drug to be delivered are known. Actuation of the device causes a mechanism to open a blister so that when the patient inhales, air is drawn through the blister entraining the dose therein which is then carried out of the blister through the device and via the patient's airway down into the lungs.

It is advantageous for the inhaler to be capable of holding a number of doses to enable it to be used repeatedly over a period of time without the requirement to open and/or insert a blister into the device each time it is used. Therefore, many conventional devices include means for storing a number of blisters each containing an individual dose of medicament. When a dose is to be inhaled, an indexing mechanism moves a previously emptied blister away from the opening mechanism so that a fresh one is moved into a position ready to be opened for inhalation of its contents.

In one embodiment, the inhalation device has a simple construction and is capable of storing a relatively large number of blisters that are also capable of containing a large payload without any significant increase in the overall size of the device. The inhalation device should also be easy to make, assemble and operate, as well as being cheap to manufacture.

More specifically, the device comprises a housing to receive a plurality of blisters, for example in a strip, each having a puncturable lid and containing a dose of medicament for inhalation by a user, a mouthpiece through which a dose of medicament is inhaled by a user and, an actuator operable to sequentially move each blister into alignment with a blister piercing member, said actuator also being operable to cause the blister piercing member to puncture the lid of a blister such that, when a user inhales through the mouthpiece, an airflow through the blister is generated to entrain the dose contained therein and carry it out of the blister and via the mouthpiece into the user's airway.

In a preferred embodiment, the actuator is pivotally mounted to the housing and may comprise an arm which may be pivotally mounted to the housing at one end. The blister piercing member may comprise a pair of piercing heads depending from one side of said arm positioned so as to extend through the aperture in the housing in a closed position, in which the arm lies substantially against the housing, to pierce the lid of a blister aligned with the aperture.

Each piercing head may preferably comprise a primary cutting element and a pair of secondary cutting elements extending laterally across each end of the primary cutting element. Conveniently, the primary cutting element and the secondary cutting elements each have a pointed tip, the tip of the primary cutting element extending beyond the tips of each of the secondary cutting elements. Ideally, the secondary cutting elements are parallel to each other and extend at right angles to the primary cutting element, although the secondary elements need not be parallel and could extend from the primary cutting element at any convenient angle.

In a preferred embodiment, an opening is formed in the arm in the vicinity of each piercing head, at least one of said openings forming an airflow inlet into a blister and, at least one other of said openings forming an airflow outlet from a blister. Conveniently, the secondary cutting elements upstand from the edge or periphery of said opening in the arm and the primary cutting element extends across the opening and joins each of the secondary cutting elements together.

Advantageously, the mouthpiece is on the arm and extends in a direction opposite to the direction in which the piercing heads extend, the openings in the arm being in communication with the inside of the mouthpiece. In one embodiment, the mouthpiece, the arm and the piercing heads are integrally formed, although the piercing heads may also be formed on a separate piercing module that is removably mountable on the arm or is at least separately attachable to the arm during manufacture.

The mouthpiece preferably includes a primary chamber having an outside air inlet in communication, via the primary chamber, with the or each airflow inlet opening in the arm and, a secondary chamber in communication with the or each airflow outlet opening in said arm such that, when a user inhales through the mouthpiece, air is drawn through the or each airflow inlet opening into the blister via the outside air inlet and the primary chamber to entrain the dose in the airflow, said entrained dose passing through the or each airflow outlet openings into the secondary chamber of the mouthpiece from where it is carried into the user's airway.

A partitioning wall may separate the primary and secondary chambers within the mouthpiece and at least one air bypass aperture may extend through the partitioning wall to communicate the primary chamber with the secondary chamber. As air can pass directly from the primary to the secondary chambers when a user inhales, in addition to passing through the blister, the effort required to inhale through the mouthpiece is reduced.

The or each bypass aperture may be configured such that the airflow from the primary chamber into the secondary chamber through the or each bypass aperture and the airflow from the or each airflow outlet openings meet substantially at right angles to each other. As the flows meet at an angle, the degree of turbulence is increased which assists in the deagglomeration of the dose and the creation of an inhalable aerosol.

In a preferred embodiment the inhaler includes an indexing mechanism including an indexing member that moves so as to move a blister into alignment with the blister piercing member. Most preferably, the indexing member is a wheel which rotates so as to move a blister into alignment with the blister piercing member. However, it is also envisaged that other arrangements are possible such as, for example, a mechanism that incorporates a sliding or reciprocating member.

In a preferred embodiment, the inhaler is configured so that indexing of the blister strip occurs when the actuator is pivoted in one direction and piercing of a blister occurs when it is rotated in the opposite direction. However, the device can also be configured so that the indexing wheel rotates, to move a blister into alignment with said blister piercing member, in response to rotation of the actuator with respect to the housing in one direction, movement of the actuator in the same direction also being operable to puncture the lid of a blister aligned with the blister piercing member.

Preferably the indexing wheel and the actuator include co-operating means thereon that engages when the actuator is rotated in one direction to cause rotation of the indexing wheel.

In one embodiment, the cooperating means comprise a set of ratchet teeth on the indexing wheel and a drive pawl on the actuator.

Advantageously, means depend from the housing to substantially prevent rotation of the indexing wheel other than by movement of the actuator in said one direction.

In one embodiment said means comprises a first resiliently deformable anti-rotation pawl on the housing that extends into one of said recesses in the indexing wheel, the actuator including means for deflecting the first anti-rotation pawl from the recess to permit rotation of the indexing wheel when the drive pawl engages with the ratchet teeth.

The actuator may include a drive plate and the means on the actuator for deflecting the first anti-rotation pawl comprises a release pin upstanding from the drive plate that engages with and resiliently deflects the pawl out of the recess to allow rotation of the indexing wheel.

The inhaler may also comprise a second resiliently deformable anti-rotation pawl on the housing and a cam member on the actuator, the cam member engaging with a cam surface on the second anti-rotation pawl when the first anti-rotation pawl is deflected out of a recess to prevent rotation of the indexing wheel through more than a predetermined angle.

The inhaler may include a cap attached to the housing pivotable between a closed position in which it covers the actuator and mouthpiece and an open position in which the actuator and mouthpiece are revealed to enable a user to inhale through the mouthpiece.

In another embodiment of the invention, the indexing wheel rotates to move a blister into alignment with the blister piercing member in response to rotation of the cap with respect to the housing from the open to the closed position. This embodiment simplifies the operation of the device even further by providing that the piercing and indexing steps are performed in response to opening and closing of the cap that locates over the mouthpiece.

Preferably, the cap and the actuator include co-operating means to couple the actuator to the cap such that the actuator rotates relative to the housing in response to rotation of the cap between the open and closed positions.

The cooperating means may comprise a cam guide slot on the cap and a cam follower on the actuator slideably located within the cam guide slot. Ideally, the cam guide slot is shaped such that when the cap is rotated from its closed to its open position, the cam follower travels along the cam guide slot to rotate the actuator and cause the blister piercing member to pierce a blister aligned therewith the aperture and, when the cap is rotated from its open to its closed position, the cam travels back along the cam guide slot to cause the actuator to rotate in the opposite direction and withdraw the piercing member from the blister. Furthermore, the cam guide slot may be configured so that the actuator does not rotate until towards the end of the movement of the cap from its closed to its open position and rotates at the beginning of the movement of the cap from its open to its closed position.

In a preferred arrangement, the indexing wheel and the cap each include a toothed gear member mounted thereon engaged such that rotation of the cap between the open and closed positions causes rotation of the gear member on the indexing wheel.

A clutch member preferably couples the gear member on the indexing wheel to the indexing wheel such that the indexing wheel rotates together with the gear member coupled thereto when the cap is rotated from the open to the closed position to move a subsequent blister into alignment with the blister piercing member.

The housing advantageously includes a chamber to receive used blisters. The chamber may be covered by a lid attached to the housing which is openable to facilitate removal of a portion of used blisters from the blisters remaining in the device.

In one embodiment, a separating element is mounted on the housing, which is operable to enable detachment of said portion of used blisters. The separating element preferably includes a resilient blister grip that is operable to press a blister strip against the housing to facilitate separation of said portion from said remaining blisters.

The inhaler according to the invention may also incorporate a coiled strip of blisters, each having a puncturable lid and containing a dose of medicament for inhalation by a user, located in the housing.

Using an inhaler as described herein may include the step of rotating the actuator to move a blister into alignment with a blister piercing member in the housing and to puncture the lid of a blister aligned with the blister piercing member and, inhaling through the mouthpiece to generate an airflow through the blister to entrain the dose contained therein and carry it through the aperture and via the mouthpiece into the user's airway.

The step of rotating the actuator may include the step of rotating it in a first direction to puncture the lid of a blister aligned with the blister piercing member and, once the inhalation step is complete, rotating it in a second direction to move a subsequent blister into alignment with the blister piercing member in the housing. Additionally, the step of rotating the actuator may comprise the step of rotating a cap coupled to the actuator.

According to another aspect of the invention, there is provided an inhaler comprising a housing to receive a blister having a puncturable lid and containing a dose of medicament for inhalation by a user, the device comprising a piercing head for puncturing the lid of a blister so that the dose contained therein can be inhaled by the user from the blister through the device, wherein the piercing head comprises a primary cutting element which is configured to cut, as the piercing head enters the blister, a first linear slit in the lid and, secondary cutting elements extending laterally from the primary cutting element which are configured to cut, as the piercing head continues to enter the blister, second linear slits that extend across each end of the first linear slit formed by the primary cutting element, the primary and secondary cutting elements together forming a pair of flaps in the lid which are folded aside by the piercing head upon further entry of the piercing head into the blister.

The inhaler may be capable of receiving just a single blister. However, in a preferred embodiment, it receives a strip of blisters each containing a dose of medicament. In this case, the inhaler may include a blister strip indexing mechanism, such as those described with reference to other embodiments of the invention, which is operable to cause the blister strip to sequentially index the blisters into a position in which each blister will be pierced by the piercing head.

In a preferred embodiment, the piercing head comprises a pair of secondary cutting elements. The secondary cutting elements may be spaced from each other and the primary cutting element is mounted on and extends between said pair of secondary cutting elements.

Preferably, the primary cutting element is formed from a blade, the plane of the blade lying substantially at right angles to a plane occupied by the lid of a blister, which is located in the inhaler in a position ready for piercing.

The primary cutting element advantageously has a sharpened edge for cutting the first linear slit in the lid of the blister. The edge may taper towards a pointed tip which may be located midway between the secondary cutting elements.

The secondary piercing elements are positioned so that they each extend laterally across either end of the primary piercing element.

Each of the secondary piercing elements may be formed from a blade, the plane of the blade lying substantially at right angles to the plane of the blade forming the primary piercing element and at right angles to the lid of a blister located in a piercing position. As with the primary piercing element, each of the secondary piercing elements may have a sharpened edge to cut the second linear slits in the lid of a blister.

The edge of each of the secondary piercing elements tapers to a pointed tip.

In a preferred embodiment, the pointed tip of each of the secondary piercing elements lie in the plane occupied by the primary piercing element.

Conveniently, the pointed tip of each of the secondary piercing elements lies at the same height as the primary piercing element at the point at which the primary piercing element and secondary piercing element meet each other.

In another embodiment, the primary cutting element divides each secondary cutting element into first and second cutting members that extend laterally from opposite sides of the primary cutting element.

Preferably, the first and second cutting members converge towards each other at an angle and the primary cutting element upstands from the top of the secondary cutting members from a point on each secondary cutting element at which the first and second cutting members meet.

The secondary cutting elements may be angled inwardly towards each other to assist in the formation and folding of the flaps in the lid of the blister as the piercing head enters the blister.

The inhaler preferably comprises a pair of piercing heads upstanding from a piercing member.

Preferably, the primary and secondary cutting elements are integrally moulded in one piece.

In a preferred embodiment, the secondary cutting elements extend laterally from the primary cutting element at an angle of 90 degrees to the primary cutting element. However, it is also envisaged that the secondary cutting elements may extend laterally from the primary cutting element at an angle of less than, or more than, 90 degrees.

The primary cutting element preferably divides each of the secondary cutting elements into secondary cutting members that extend laterally from the primary cutting element by different distances so that the flap cut in the lid of a blister by the secondary cutting members extending laterally from one side of the primary cutting element is of a different size to the flap cut in the blister by the secondary cutting members that extend laterally from the other side of the primary cutting member.

According to any of the embodiments of the invention, the piercing member may comprise a discrete piercing module which is moulded separately and then subsequently attached to the actuator either permanently during assembly or so that it may be removed from the actuator by the user for replacement, if necessary. The piercing module conveniently comprises a main body portion with first and second piercing heads upstanding therefrom.

Preferably, an air inlet and an air outlet aperture extends through the main body portion of the piercing module, one of the piercing heads depending from the periphery of the air inlet and extending over the air inlet and the other piercing head depending from the periphery of the air outlet and extending over the air outlet.

The main body portion may include a recessed region around the air inlet, the piercing head depending from the periphery of the air inlet from the recessed region.

The air outlet aperture is preferably in communication with an air outlet tube extending from the main body in an opposite direction to the piercing head extending from the periphery of the air outlet aperture.

In a preferred embodiment, the air outlet tube comprises axially extending ridges formed on its outer surface, which locate the piercing head within a walled recess in the mouthpiece.

A space formed between the ridges and the walled recess advantageously comprises a bypass air conduit for the direct flow of air into the mouthpiece from outside when a patient inhales through the mouthpiece.

In a preferred embodiment, the indexing mechanism comprises a blister strip locator chassis defining a path for the strip of blisters past the aperture in the housing.

Preferably, a resiliently deformable arm extends from the blister strip locator chassis and the indexing mechanism comprises an indexing wheel rotatably mounted to the free end of the resiliently deformable arm over which a strip of blisters is passed.

The indexing wheel may comprise a set of spokes and the actuator includes a drive tooth engageable with a first spoke when the actuator is pivoted relative to the housing into an open position to cause the indexing wheel to rotate together with the actuator to index the blister strip.

Preferably the inhaler includes an anti-rotation ramp on the housing which is engaged by another spoke of the indexing wheel when the indexing wheel rotates thereby causing the arm to deform to allow said spoke to clear the anti-rotation ramp, the arm returning to its undeformed state once the spoke has cleared the ramp, thereby preventing rotation of the indexing wheel in the opposite direction.

Preferably, the drive tooth on the actuator is shaped so that, when the actuator is rotated in the opposite direction from its open into its closed position, the drive tooth slides over the top of the preceding spoke of the indexing wheel.

Conveniently, the edge of each spoke is shaped to allow the drive tooth to pass over it when the actuator is pivoted from its open into its closed position.

In one embodiment, a location ramp may be positioned adjacent to but spaced from the anti-rotation ramp. In this case, the drive tooth may be operable to cause the arm to resiliently deform as the drive tooth slides over the top of the spoke to cause another spoke of the indexing wheel to extend into the space between the anti-rotation and location ramps and prevent rotation of the indexing wheel in either direction.

Embodiments of the invention will now be described, by way of example only, with reference to the accompanying drawings, in which:—

FIG. 1 is a perspective view of an inhaler according to an embodiment of the invention;

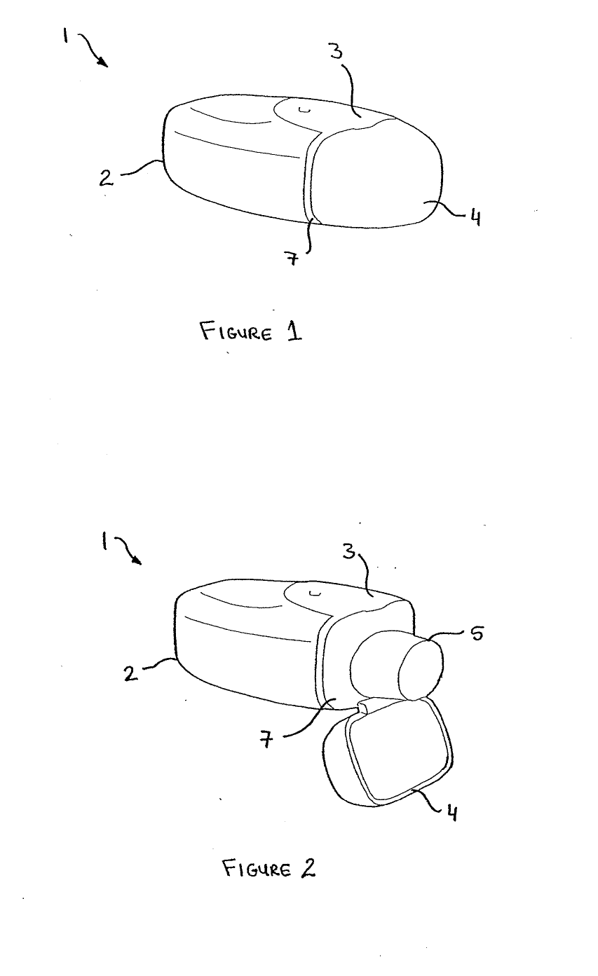

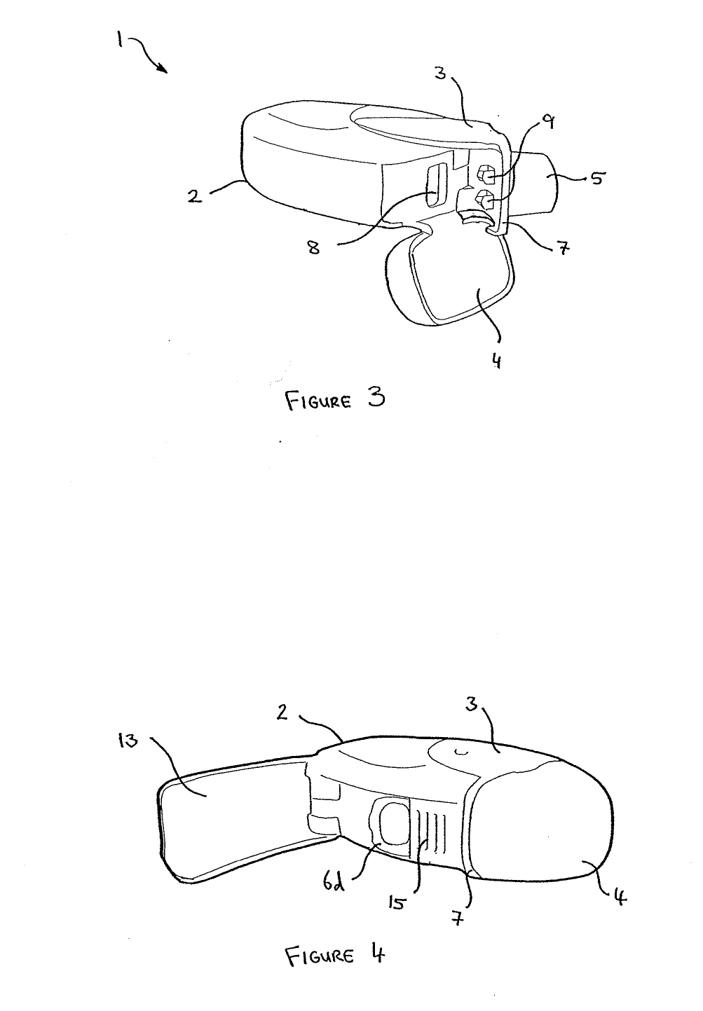

FIG. 2 is a perspective view of the inhaler illustrated in FIG. 1 with the cap open to reveal the mouthpiece and the actuator in a closed position;

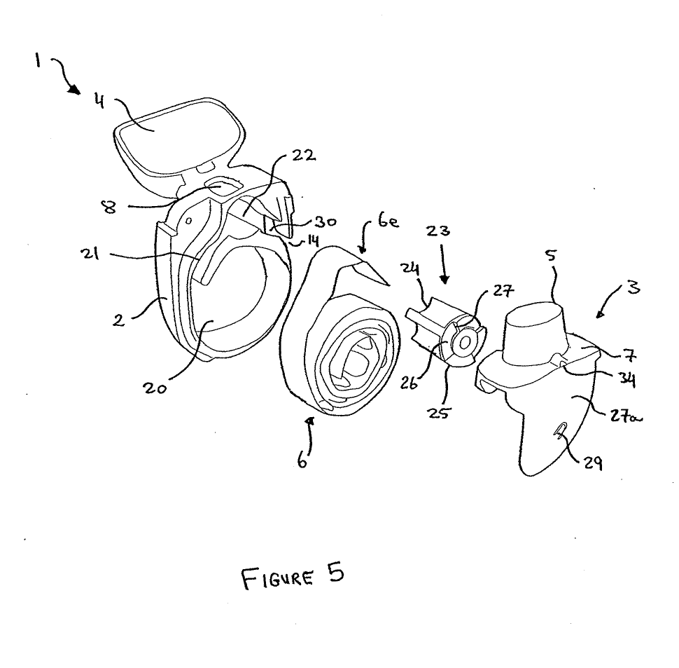

FIG. 3 is a perspective view of the inhaler illustrated in FIG. 2 with the actuator in an open position;

FIG. 4 is a perspective view of the inhaler shown in FIG. 1 with a used blister chamber cover open;

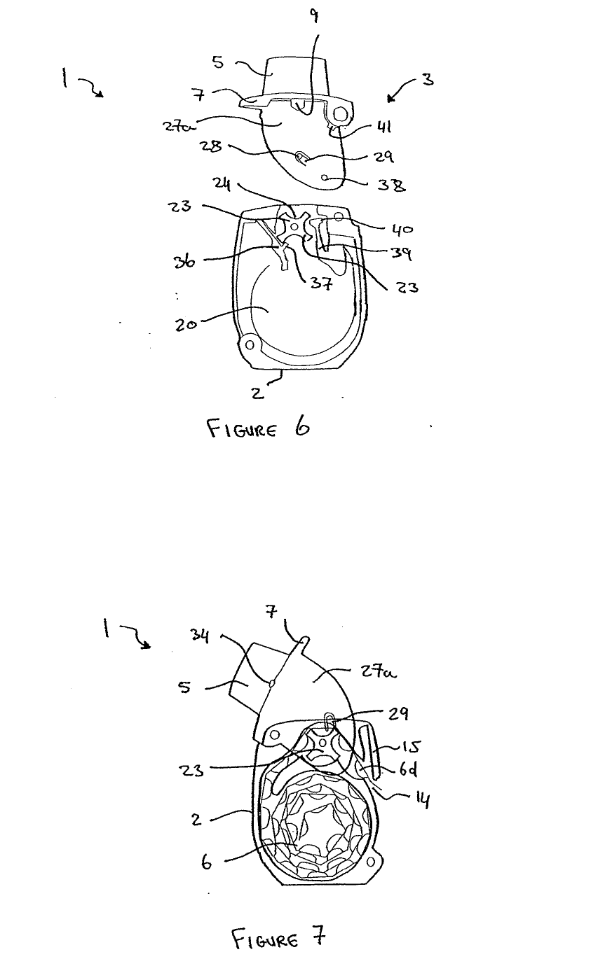

FIG. 5 is an exploded perspective view of the inhaler illustrated in FIGS. 1 to 4 also showing a coiled strip of blisters used with the device according to the invention;

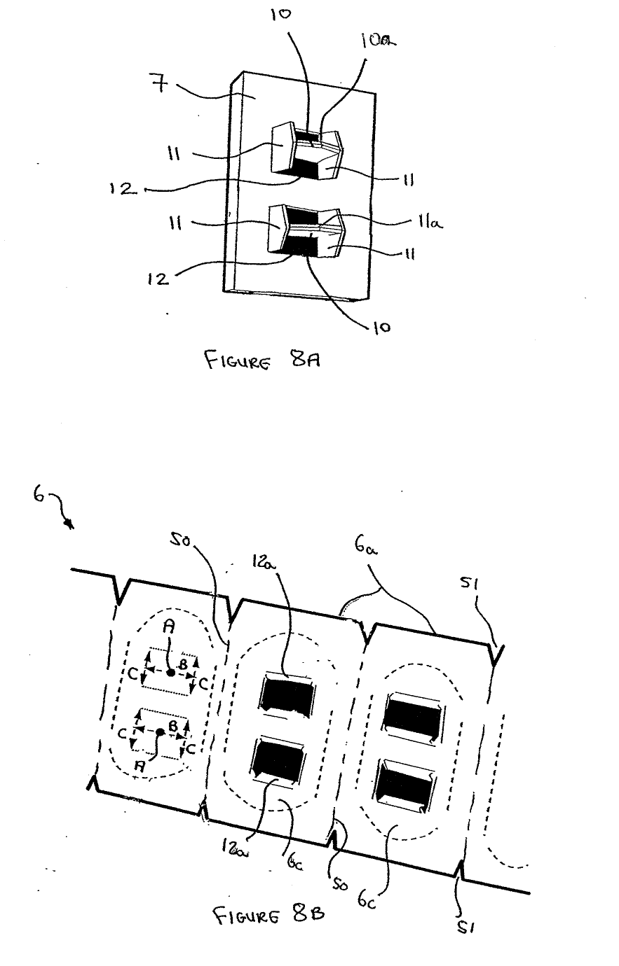

FIG. 6 is a rear cross-sectional view of the inhaler illustrated in FIGS. 1 to 5 with the actuator shown separately;

FIG. 7 is a front cross-sectional view of the inhaler illustrated in FIG. 6 in which the actuator is pivotally mounted to the housing;

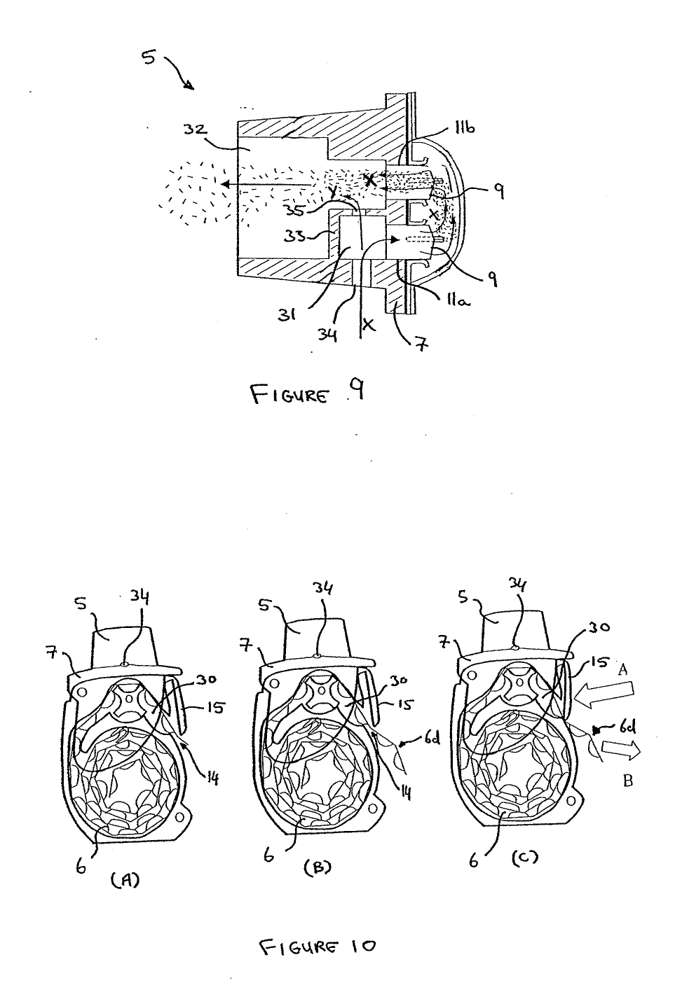

FIGS. 8A and 8B shows the configuration of the piercing elements on the actuator and a small portion of a strip of blisters to illustrate the type of cut made therein by the piercing elements, respectively;

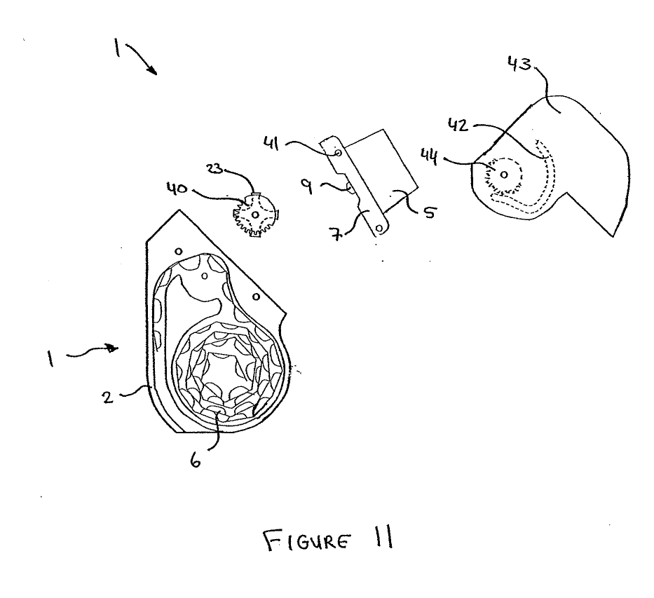

FIG. 9 is a side sectional view of the mouthpiece and actuator during inhalation from a blister;

FIG. 10A to 10C show a series of front cross-sectional views of the inhaler according to the invention with a blister strip located therein to show the path of used blisters from the housing;

FIG. 11 is an exploded side cross-sectional view of an inhaler according to another embodiment of the invention;

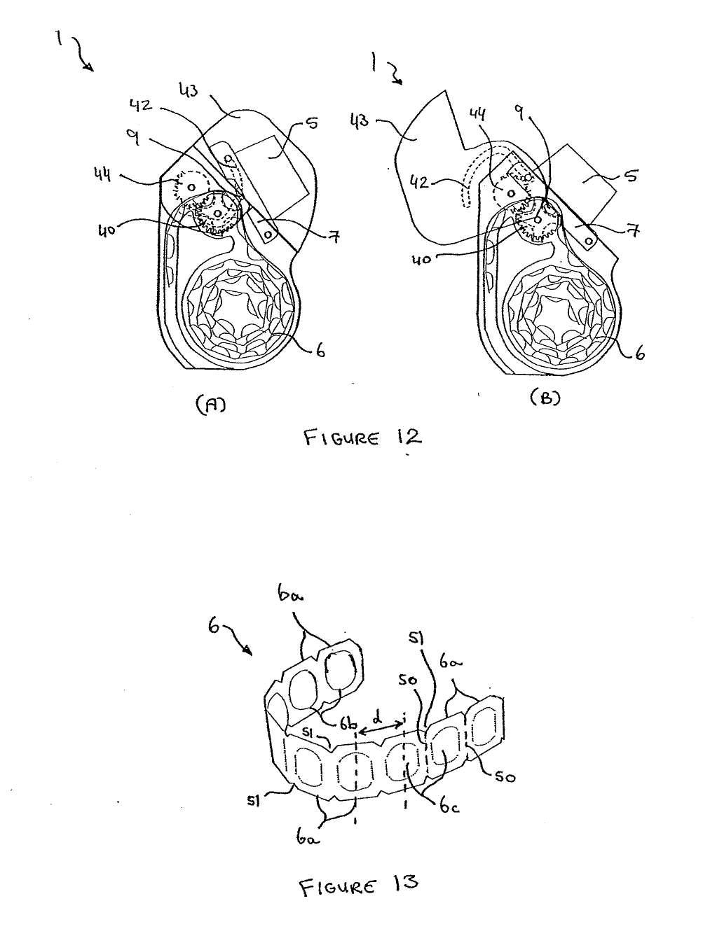

FIGS. 12A and 12B are side cross-sectional views of the inhaler according to the second embodiment with the cap in the closed and open positions respectively;

FIG. 13 shows a short portion of a strip of blisters for use in the inhaler according to any embodiment of the invention;

FIGS. 14A and 14B are perspective views of another embodiment of inhaler according to the present invention;

FIGS. 15A and 15B show a side cross-sectional view of the inhaler illustrated in FIGS. 14A and 14B with the actuator in a closed and open position respectively.

FIG. 16 is another side cross-sectional view of the inhaler shown in FIGS. 14A and 14B;

FIG. 17 is a side sectional view of the mouthpiece and actuator during inhalation from a blister;

FIG. 18 shows an alternative configuration of piercing elements on the actuator according to any embodiment of the invention, and

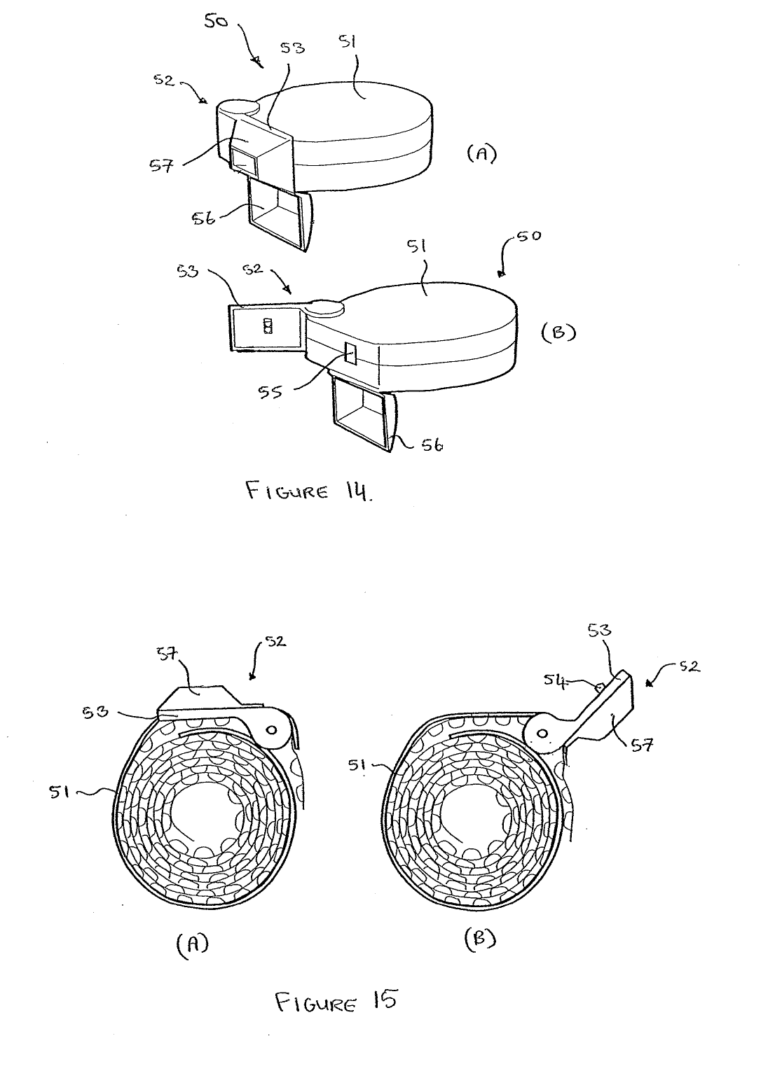

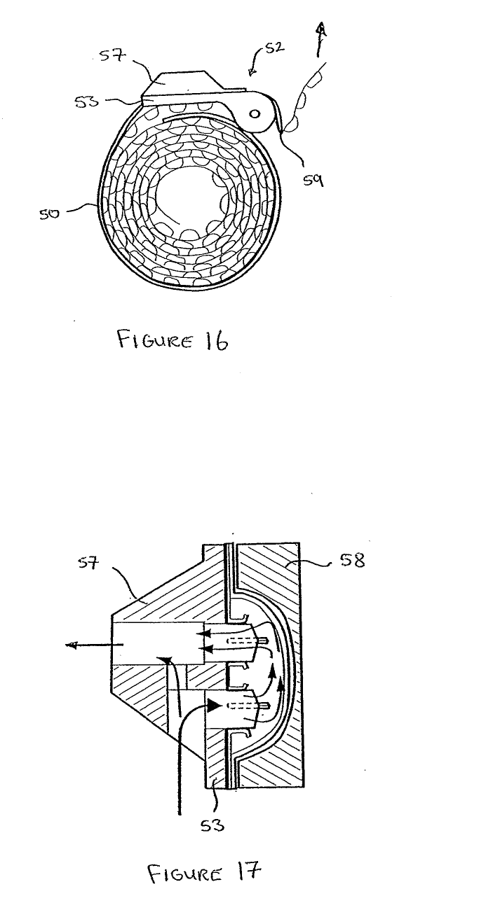

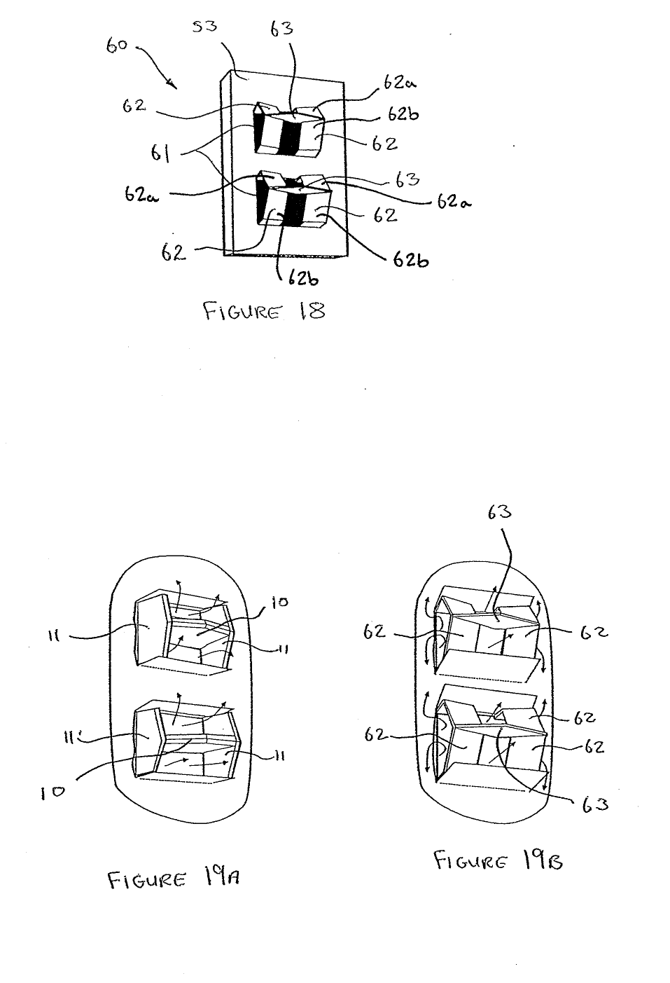

FIG. 19A shows the airflow into the blister using the piercing elements of FIG. 8A and FIG. 19B shows the airflow into the blister using the piercing element of FIG. 18.

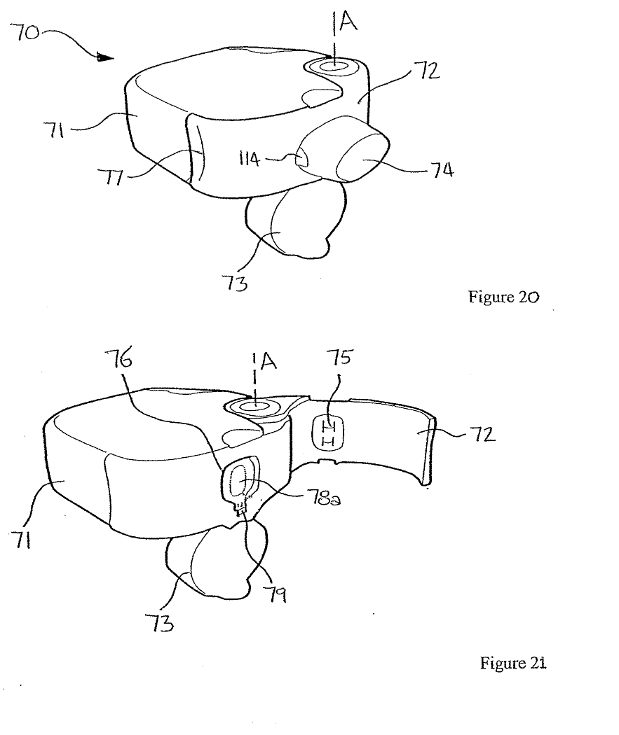

FIG. 20 illustrates a perspective view of another embodiment of inhaler according to the present invention with the cap open and the actuator in the closed position in which it lies against the housing of the inhaler;

FIG. 21 illustrates a perspective view of the inhaler shown in FIG. 20 but after the actuator has been pivoted with respect to the body into an open position;

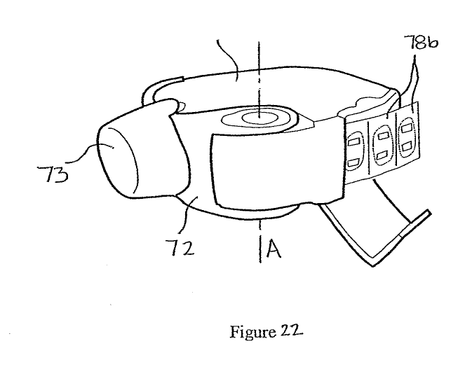

FIG. 22 illustrates another perspective view of the inhaler shown in FIGS. 20 and 21 with a strip of used blisters protruding from the housing and a used blister door in an open position;

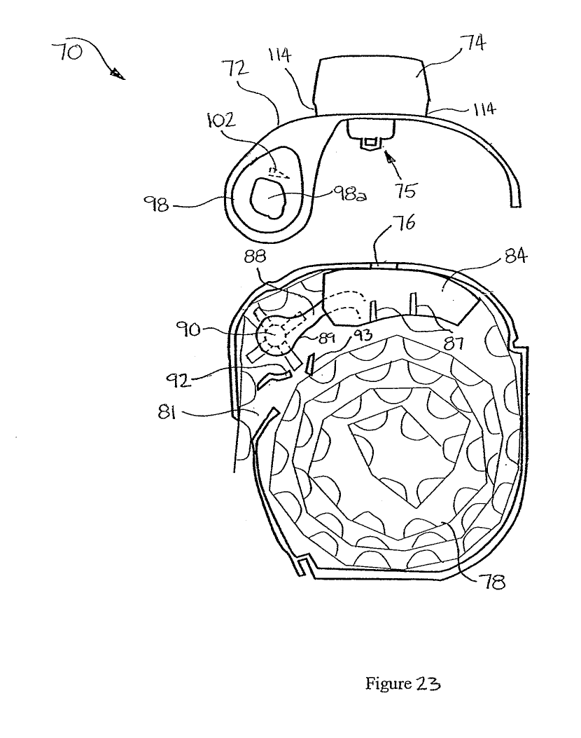

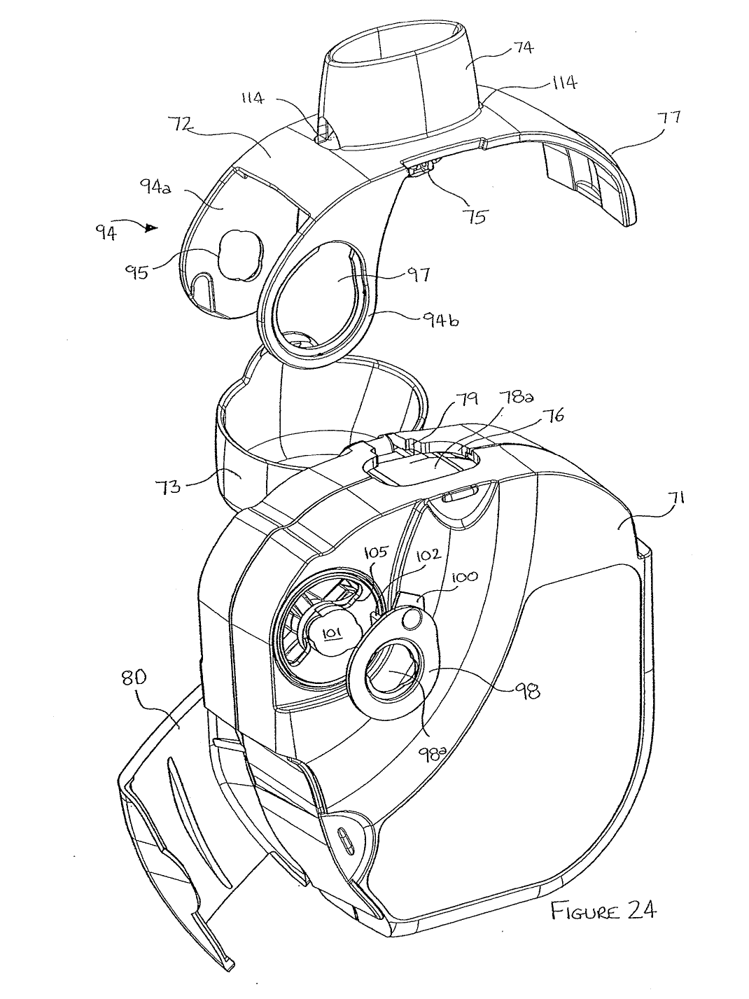

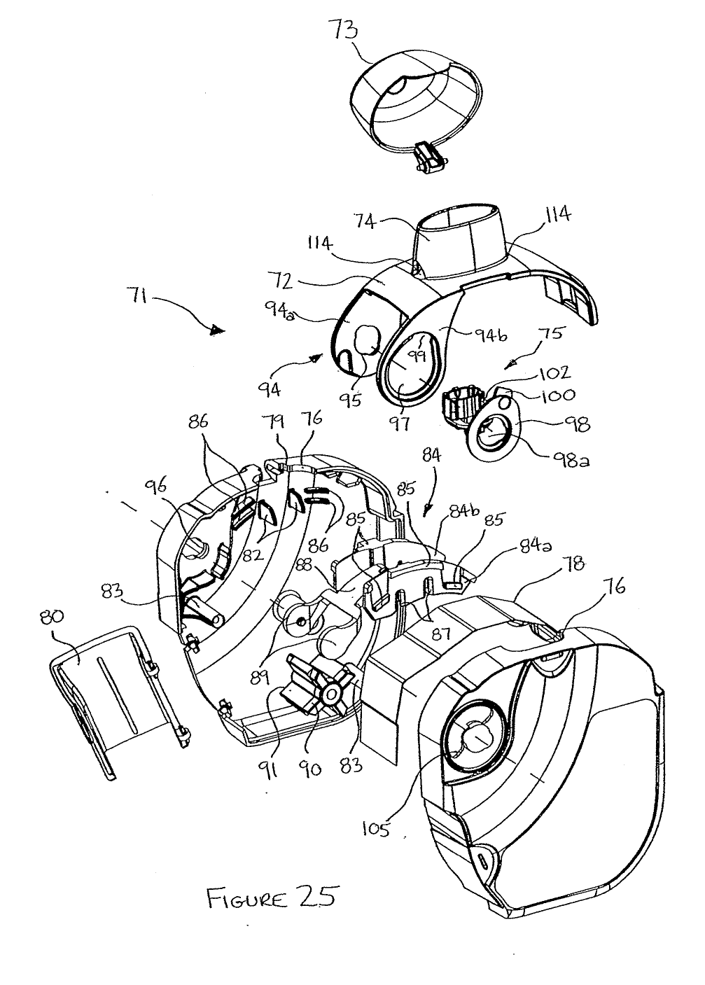

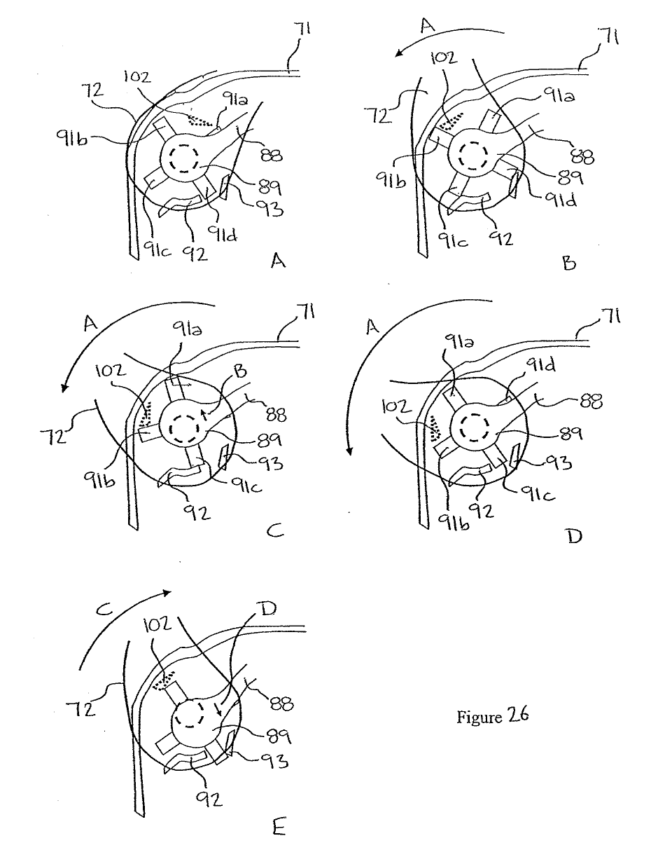

FIG. 23 illustrates a side view of the inhaler shown in FIGS. 20 to 22 with one half of the housing omitted so that the internal components are visible together with a coiled strip of blisters located in the housing, the actuator is shown detached from the housing and the used blister cover is omitted altogether for clarity;

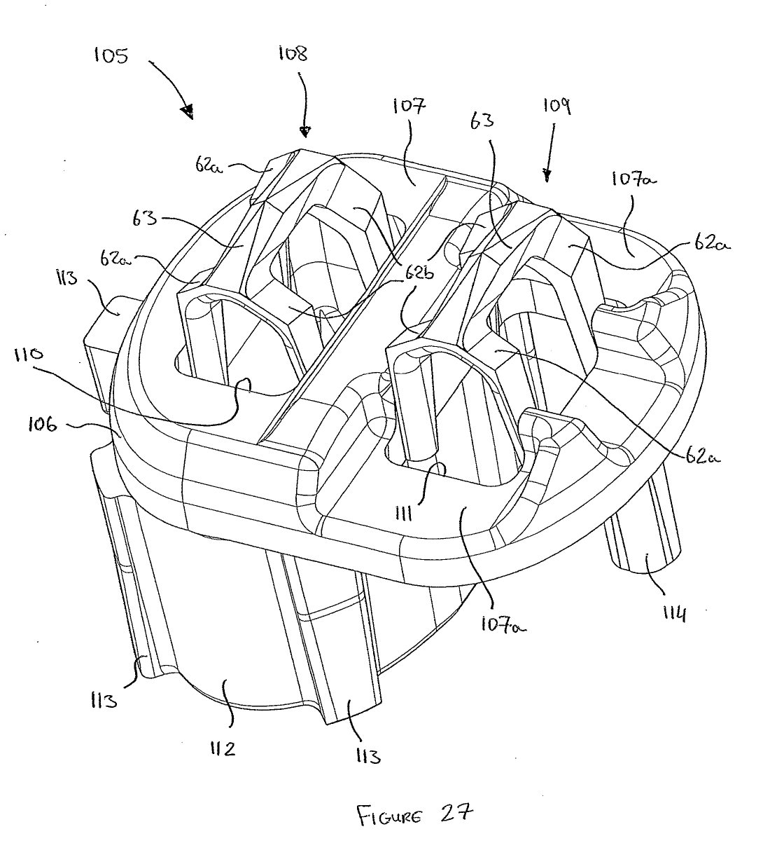

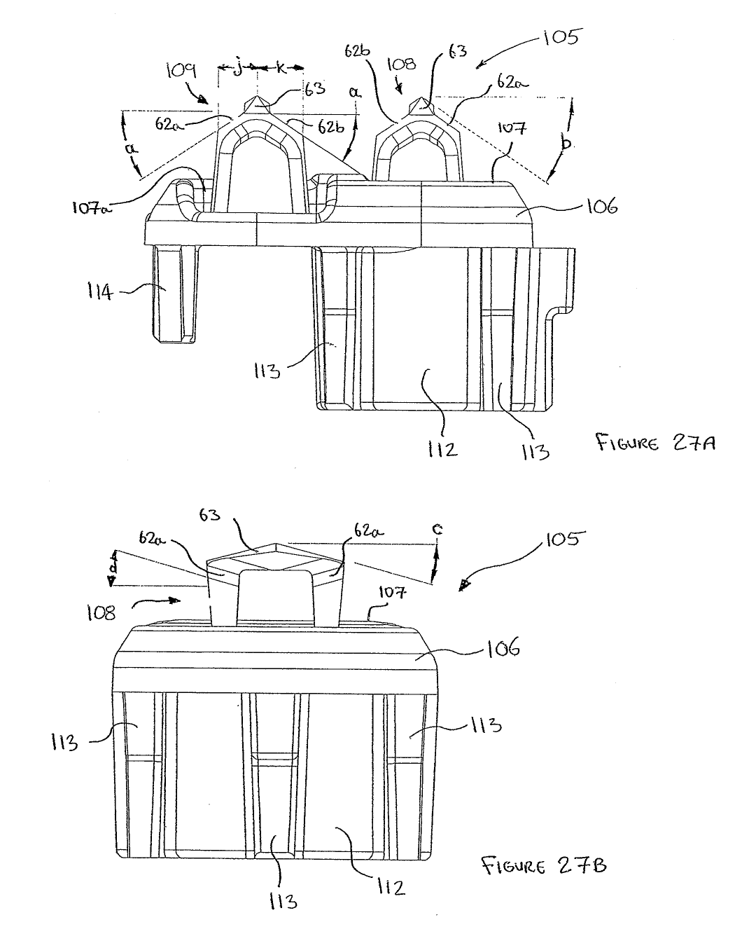

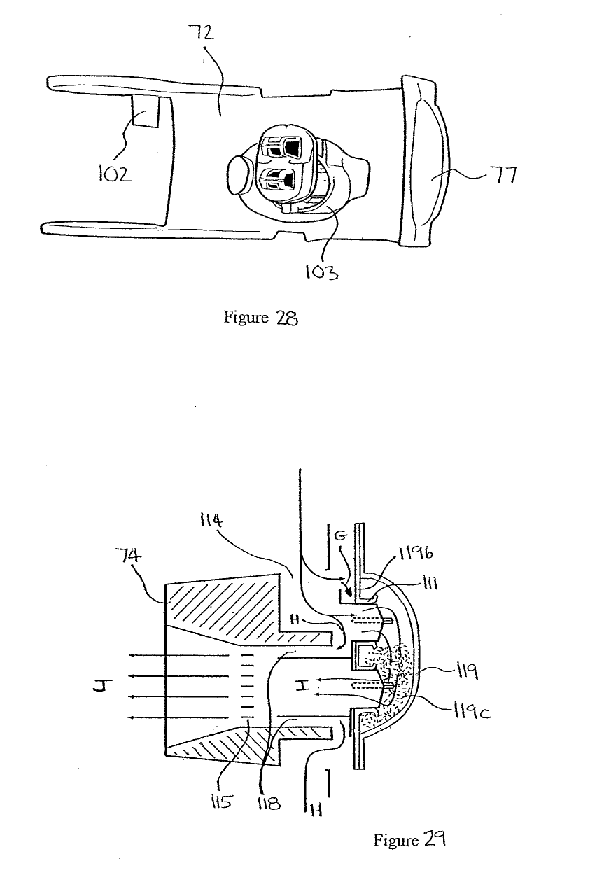

FIG. 24 illustrates a partially exploded perspective view of the inhaler shown in FIGS. 20 to 23;

FIG. 25 illustrates a fully exploded perspective view of the inhaler shown in FIG. 24;

FIG. 26A to 26E each illustrate an enlarged portion of the inhaler shown in FIG. 23 and show the various positions of the indexing wheel during operation of the device;

FIG. 27 illustrates a perspective view of a piercing head module primarily intended for use with the embodiment described with reference to FIGS. 20 to 27 but which may also be used with any of the previously illustrated embodiments;

FIG. 27A illustrates a side view of the piercing head module shown in FIG. 27;

FIG. 27B illustrates an end view of the piercing head module shown in FIGS. 27 and 27A;

FIG. 28 illustrates a perspective view of the actuator used with the embodiment shown in FIGS. 20 to 26 with the piercing head module of FIG. 27 mounted thereto;

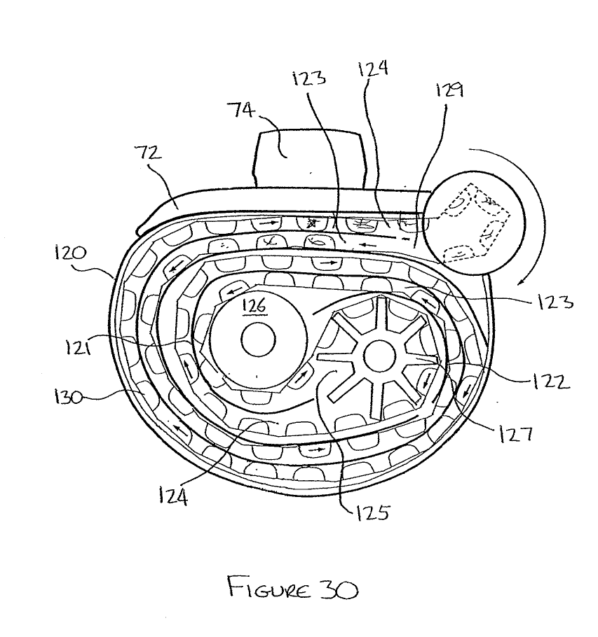

FIG. 29 is a side sectional view to show the passage of air through the piercing head module of FIG. 27, and

FIG. 30 is a side view of an inhaler having an endless loop drive according to another embodiment of the invention with one half of the housing removed to reveal the internal components.

A first embodiment of the inhaler according to the invention will be described with reference to FIGS. 1 to 10. This embodiment provides a simple, easy to use inhalation device that indexes and pierces a blister using the same actuator. Furthermore, the actuator both indexes and pierces a blister during the same stroke or direction of rotation of the actuator.

Referring now to the drawings, there is shown in FIG. 1 an inhaler 1 according to a first embodiment of the invention comprising a housing 2 to which is pivotally mounted an actuator 3. A cap 4 is integrally hinged to the top edge of the housing 2 and is pivotable between a closed position, as shown in FIG. 1, to an open position, as shown in FIG. 2, to gain access to a mouthpiece 5 integrally formed with and upstanding from the actuator 3. The cap 4 completely covers and protects the mouthpiece 5 when closed and prevents contamination thereof or the possible ingress of dirt into the housing 2 which could otherwise be inhaled when the device is used.

The inhaler 1 is intended for use with a strip 6 of moisture proof blisters (see FIG. 13) each containing a pre-measured dose of powdered medicament for inhalation. Each blister 6a in the strip 6 comprises a generally hemispherically shaped pocket 6b and a flat puncturable lid 6c permanently heat sealed to the pocket 6b to hermetically seal the dose therein. The strip 6 is preferably manufactured from foil laminate or a combination of foil laminate, such as aluminium, and plastics material.

In a preferred embodiment the blisters consist of a base and a lid. The base material is a laminate comprising a polymer layer in contact with the drug, a soft tempered aluminium layer and an external polymer layer. The aluminium provides the moisture and oxygen barrier, whilst the polymer provides a relatively inert layer in contact with the drug. Soft tempered aluminium is ductile so that it can be “cold formed” into a blister shape. It is typically 45 μm thick. The outer polymer layer provides additional toughness to the laminate. The lid material is a pierceable laminate comprising a heat seal lacquer, a hard rolled aluminium layer (typically 20-30 μm thick) and an external lacquer layer. The heat seal lacquer bonds to the polymer layer of the base foil laminate during heat sealing. Materials for the polymer layer in contact with the drug include poly vinyl chloride (PVC), polypropylene (PP) and polyethylene (PE). In the case of PE, the heat seal lacquer on the foil lid is replaced with a further layer of PE. On heat-sealing, the two layers of PE melt and weld to each other. The external polymer layer on the base foil is typically oriented polyamide (oPA).

The actuator 3 comprises a lever arm 7 having one end pivotally mounted to the housing 2 to enable it to rotate from a closed position shown in FIGS. 1, 2 and 4 into an open position shown in FIG. 3. As can be seen from FIG. 3, the housing 2 has an aperture 8 therein to receive a piercing member comprising a pair of piercing heads 9 that extend from the lever arm 7 when the actuator 3 is in a closed position and penetrate the lid 6c of a blister located within the housing 2 immediately behind the aperture 8.

The shape of the piercing heads 9 will now be described with reference to FIG. 8A. This is important because the openings that are made in the lid 6c of a blister 6a must be of a sufficient cross-sectional area and shape to promote the free-flow of air through the blister 6a and to ensure that all of the internal volume of the blister 6a is swept by the airflow and consequently that all, or substantially all, of the dose is entrained and carried out of the blister 6a. Each piercing head 9 comprises a generally “H” shaped element having a flat blade-like central tooth or primary cutting element 10 and a pair of flat blade-like end teeth or secondary cutting elements 11 extending laterally across each end of the primary piercing element 10. Each of the primary and secondary cutting elements 10,11 taper to a pointed tip. The pointed tip 10a of the primary cutting element 10 may be located in its centre i.e. midway between the secondary cutting elements 11. However, it may be advantageous to form the primary cutting element 10 so that its pointed tip 10a is closer to one of the secondary piercing elements 11 than the other secondary cutting element 11, for example in order to facilitate correct piercing when the angle of approach of the piercing heads 9 is not normal to the foil. The height of each of the secondary cutting elements 11 is such that the pointed tips 11a of the secondary cutting elements 11 are at the same height as the edges of the primary cutting element 10 where the primary and secondary cutting elements 10,11 meet each other. The pointed tip 10a of the primary cutting element 10a is therefore above the pointed tip 11a of each of the secondary cutting elements 11 so that the primary cutting element 10 slits, or has at least initiated, the first linear slit in the blister before either of the secondary cutting elements 11 begin to cut the second linear slits in the blister. The top edges of each primary and secondary cutting elements 10,11 are sharpened to enable them to easily penetrate and cut the lid 6c of a blister 6a.

As can be seen in FIG. 8A, the secondary cutting elements 11 of each piercing head 9 upstand from opposite edges of an aperture 12 in the lever arm 7 to enable the flow of air through the arm 7 into and out of the blister 6b via the holes made in the lid 6c of the blister 6b with the piercing members 9. The primary cutting element 10 is attached to, and is supported between, each of the secondary cutting elements 11 and the primary cutting element extends across the aperture 12 and so is not attached directly to the lever arm 7.

FIG. 8B illustrates a short section of a strip 6 of blisters 6a to show the shape and size of the openings that each of the piercing elements 9 described with reference to FIG. 8A cut in the lid 6c of a blister 6b. The primary cutting elements 10 penetrate the lid 6c first (point A in FIG. 8B) and, as they enter the blister 6a, two linear cuts or slits are made by each of them, as indicated by arrows “B”. As the piercing head further enters the blister, the secondary cutting elements 11 penetrate the blister 6a and further linear cuts are made at each end of the linear cuts perpendicular to the first linear cut formed by the primary piercing element 10, as indicated by arrows “C”. These cuts have the effect of creating flaps 12a that are folded back into the blister 6a as the piercing head 9 enters further into the blister. These piercing heads 9 are capable of forming openings that extend to over 30 to 50% of the surface area of a lid 6c of a blister 6a. For example, in the embodiment of FIG. 27, the blister lid area is 67 mm² and the piercers open an area of 29 mm² which is equivalent to 43% of the surface area of the lid.

As shown in FIG. 4, a cover 13 is pivotally attached to the side of the housing 2 and encloses a space to receive used blisters 6d that are fed into said space through a slot 14 in the wall of the housing 2. The space within the cover 13 is large enough to accommodate only a few used blisters 6d therein and so a resiliently flexible blister grip 15 extends from the housing 2 and facilitates removal of some of the used blisters 6d from the blisters 6 that remain in the housing 2. To remove a section of used blisters 6d, the blister grip 15 is pressed against the strip 6 to sandwich it between the blister grip 15 and the sidewall of the housing 2. The visible section of used blisters 6d can then be grasped in the hand, torn off and discarded without inadvertently placing undue force on the remaining part of the blister strip 6 that would tend to pull it out of the housing 2. FIGS. 10A to 10C show three front cross-sectional views through the inhaler 1. In FIG. 10A, there are no empty blisters 6d protruding through the slot 14. In FIG. 10B, the device has been activated twice more and so two empty blisters 6d have now passed through the slot 14. In FIG. 10C, the blister grip 15 has been pressed against the housing 2 in the direction of arrow “A” to enable the two empty blisters 6d to be detached by pulling them in the direction of arrow “B”.

It will be appreciated that a cover 13 is not essential and the used blisters 6d may be removed as soon as they emerge from the aperture 14 in the wall of the housing 2. In another embodiment, the inhaler 1 may be provided with a cutting implement (not shown) such as a blade or serrations against which the section of used blisters 6d to be removed may be pressed to facilitate their detachment. In a preferred arrangement, a blade may be mounted to and extend from the blister grip 15 so that when it is pressed against the housing 2 it cuts the strip 6d located between the blister grip 15 and the housing 2. In yet another embodiment, the inhaler 1 may incorporate a larger chamber possibly with a take-up spool around which the used blister strip 6d may be wound so that it can be removed as a whole from the device and so avoid the need to detach sections of the strip 6d as each short section of blisters 6a are used up. However, in order to keep the device as small as possible, it is preferable to provide an arrangement in which at least some of the used blisters 6d can easily be removed from the device whilst unused blisters remain in it.

Referring now to FIG. 5, the housing 2 comprises a generally cylindrically shaped chamber 20 to receive a coiled or wound strip of blisters 6 each containing a pre-measured dose of medicament to be delivered using the inhaler 1. The leading end 6e of the strip 6 is received in a blister feed inlet path 21 which opens up into a generally cylindrical cavity 22 adjacent to and in communication with the aperture 8 in the housing 2 and in which is rotatably received an indexing wheel 23. A used blister feed outlet path 30 extends from the cylindrical cavity 22 and leads to the aperture 14 in the wall of the housing 2.

The chamber 20 has a cover (not shown in FIG. 5) that forms part of the housing 2. Preferably, the cover is removably attached to the remainder of the housing 2 to enable access to the inside of the inhaler 1 to be obtained to enable a fresh strip 6 of blisters to be inserted therein. However, it is envisaged that the device could form a disposable unit in which case a strip of blisters 6 could be mounted in the device during assembly and the cover permanently attached so that once the strip has been exhausted, the whole device is thrown away. The simplicity of the construction of the device and the relatively few separate components make the device very cheap to manufacture and so a disposable unit is a viable proposition.

The indexing wheel 23 is a generally cylindrically shaped member with a set of blister receiving grooves or recesses 24 extending longitudinally along its outer surface parallel to its axis of rotation. Each groove 24 is shaped so as to receive a blister 6a therein as the indexing wheel 23 rotates, as will be explained in more detail below. The recesses 24 are spaced at a pitch which is equal to the distance “d” between the centre lines of a pair of blisters, as indicated in FIG. 13, so that as the indexing wheel 23 rotates, a strip 6 extending through the blister feed path 21 and over the indexing wheel 23 is pulled so that a blister 6a locates in the recess 24 of the indexing wheel 23 situated immediately opposite the aperture 8, as will be explained in more detail below. To enable the indexing wheel 23 to rotate in response to rotation of the actuator 3 in one direction, ratchet teeth 25 are formed on one end face thereof for cooperation with the actuator 3 as will shortly be explained, each tooth 25 comprising an arcuately shaped ramp section 26 and a shoulder 27. The indexing wheel 23 is a close fit in the cylindrical cavity 22 so that the strip 6 is securely held by the indexing wheel 23 and each blister 6a is snugly received and held in the recess 24 opposite the aperture 8 whilst allowing for rotation of the indexing wheel 16 to feed the strip of blisters 6 through the device. As the indexing wheel 23 rotates, the used blisters 6d are fed out of the cavity 22 down the used blister feed path 30 and through the slot 14 out of the housing 2.

A drive plate 27a depends from a longitudinal edge of the lever arm 7 and carries a drive pawl 28 thereon for cooperation with the ratchet teeth 25 on the indexing wheel 23 during rotation of the actuator 3 from the open to the closed position. The drive pawl 28 is integrally formed in the drive plate 27a by cutting a U-shaped slot therein to form a resiliently deformable tab 29 from which the drive pawl 28 upstands.

The mouthpiece 5 is integrally formed with the lever arm 7 of the actuator 3 and upstands from one side thereof opposite to the side from which the piercing heads 9 extend. The interior of the mouthpiece 5 can be seen from the cross-sectional view of FIG. 9 and is divided into a primary and a secondary chamber 31,32 by a partitioning wall 33. An outside air inlet orifice 34 in the sidewall of the mouthpiece 5 close to where it joins or becomes the lever arm 7 is in communication with the primary chamber 31. The primary chamber 31 is also in communication with one of the apertures 11a in the lever arm 7 that is formed in the vicinity of a piercing head 9. The secondary chamber 32 makes up the main internal volume of the mouthpiece 5 and is in communication with the other aperture 11b in the lever arm 7. A bypass aperture 35 extends through the partitioning wall 33 to communicate the primary chamber 31 with the secondary chamber 32 for reasons that will become apparent.

The path of the blister strip 6 through the device and the way in which it is disposed within the chamber 20 can be most clearly seen in FIG. 7. It will be appreciated that the coils of the blister strip 6 are loosely wound in the chamber 20 so that the blister strip 6 will unwind in response to a pulling force applied to the leading edge 6e of the strip by the indexing wheel 23 as the indexing wheel 23 rotates.

To prevent rotation of the indexing wheel 23, other than due to rotation of the actuating member 3, the housing 2 is provided with an integrally formed resiliently flexible arm 36 carrying an anti-rotation pawl 37 that normally locates in one of the recesses of the indexing wheel 23 which is not occupied by a blister 6a, as shown in FIG. 6. Engagement of the pawl 37 with the indexing wheel 23 prevents the indexing wheel 23 from rotating. A release pin 38 upstands from the drive plate 27a which engages the arm 37 to push the pawl 38 out of the recess to allow rotation of the indexing wheel 23 when the actuator 3 approaches its fully open position.

When the pawl 38 is deflected from the recess 24, the blister strip 6 could be pulled from the housing 2. To prevent this, a second resiliently deformable anti-rotation pawl 39 is provided on the housing 2. The second anti-rotation pawl 39 has a cam surface 40 thereon which is engaged by a cam member 41 on the actuator 3 when the first anti-rotation pawl 37 is pushed out of the recess 24 of the indexing wheel 23. The second anti-rotation pawl 39 is therefore locked into position and protrudes into another recess 17 of the indexing wheel 23. This prevents the indexing wheel 23 from rotating by more than approximately 45 degrees and so the strip 6 can only be pulled through the device by about half a blister width.

It will be appreciated from the foregoing that the inhalation device according to this embodiment of the invention has a very simple construction with relatively few components. If the cap 4 is integrally formed with the housing 2 in a single moulding and the actuator 3 is formed together with the mouthpiece 5, the piercing heads 9, the drive plate 27a and the drive pawl 28 in another moulding, the device can be formed from as few as 4, 5 or 6 moulded plastic parts.

Operation of the inhaler 1 will now be described. When the inhaler 1 is not in use, the cap 4 and the lever arm 7 are both in a closed position in which the cap 4 covers the mouthpiece 5 and the lever arm 7 lies generally against the side of the housing 2 with the piercing heads 9 extending through the aperture 8 in the housing 2 and into a previously exhausted blister 6d lying immediately below the aperture 8 and constrained in the uppermost recess 24 of the indexing wheel 23 adjacent to the aperture 8. The first and second anti-rotation pawls 37,39 prevent rotation of the indexing wheel 23 in either direction and so locate the blister in position.

When the cap 4 is opened, the lever arm 7 can be pivoted into the position shown in FIG. 3. As the lever arm 7 pivots, the drive pawl 28 on the drive plate 27a rides up the ramp section 26 forming one of the ratchet teeth on the end of the indexing wheel 23 and so no rotation of the indexing wheel 23 occurs. Once a fully open position has been reached, as shown in FIG. 3, the drive pawl 28 has reached the end of the ramp section 26 and drops down against the face of a corresponding shoulder 27 so that as the actuator 3 is rotated back in the opposite direction from the open to the closed position, engagement between the drive pawl 28 and the shoulder 27 causes the indexing wheel 23 to rotate. It will be appreciated that if the lever arm 7 is not opened to its fullest extent before being returned to its closed position, the indexing wheel 23 will not rotate because the drive pawl 28 will not have dropped down to engage a shoulder 27 at the top of the ramp section 26.

Just before the lever arm 7 reaches its fully open position, the release pin 38 on the drive plate 27a engages with the arm 36 from which the first anti-rotation pawl 37 extends and deflects it so that the anti-rotation pawl 37 moves out of the recess 24 in the indexing wheel 23 so that the indexing wheel 23 can rotate and the strip 6 can be indexed when the lever arm 7 is rotated in the opposite direction. At the same time, the cam member 41 engages with the cam surface 40 of the second anti-rotation pawl 39 and locks it into position to ensure that the strip 6 cannot be pulled from the inhaler 1 by more than approximately half the width of a blister 6b.

As the lever arm 7 is pivoted back into its closed position, the indexing wheel 23 is rotated through 90 degrees as a result of engagement between the drive pawl 28 and the shoulder 27 on the indexing wheel 23. Whilst the lever arm 7 is rotated back into its closed position, the anti-rotation pawls 37,39 have returned to their original positions locking the indexing wheel 23 in place. This rotation of the indexing wheel 23 brings the next blister 6b into position immediately below the aperture 8 in the housing 2.

In the final stage of the return stroke of the lever arm 7 back to its closed position, the piercing heads 9 pass through the aperture 8 in the housing 2 and penetrate the to lid 6c of the blister 6a that has just been moved into position by the indexing wheel 23. The dose is now ready for inhalation, as will now be described.

When a user inhales through the mouthpiece 5, a low pressure region is created in the secondary chamber 32 causes air to be drawn through the blister 6a from the outside air inlet 34 via the primary chamber 31 and the airflow opening 11a in the lever arm 7, as indicated by arrows marked “X” in FIG. 9. This airflow through the blister 6b entrains the dose contained therein, which is carried into the secondary chamber 32 and from there into the patient's airway.

The turbulent airflow generated through the aperture 11b in the lever arm 7 around the piercing element 9 helps to deagglomerate the dose and create a respirable aerosol. The air bypass orifice 35 in the partitioning wall 33 between the primary and secondary chambers 31,32 reduces the overall pressure drop across the device and so makes it easier for the patient to inhale. It also increases turbulence in the secondary chamber 32. In a particularly preferred arrangement, the bypass orifice 35 is situated so that the airflow therethrough, indicated by arrow “Y” in FIG. 9, meets the airflow entering the secondary chamber 32 from the blister at a tangent or right angle so as to create a cyclonic effect or increase the airflow turbulence to assist deagglomeration.

Once the device has been used a number of times, the side cover 13 may be opened and the visible section 6d of used blisters may be detached from those that remain within the device as has already been explained.

A second embodiment of the inhaler according to the invention will now be described with particular reference to FIGS. 11 and 12. In this embodiment, the actuator is coupled to the cap covering the mouthpiece so that a blister is pierced when the cap is opened and indexed to move the next unused blister into position beneath the aperture in the housing when the cap is closed. This provides a device that is very simple to operate, as the user does not have to open the cap before pivoting the actuator to index and pierce a blister.

Referring to the exploded view of FIG. 11, the inhaler 1 is similar to the device described with reference to the first embodiment except that the ratchet teeth on the indexing wheel 23 have been replaced with a toothed gearwheel 40 which is attached to the indexing wheel via a one-way or clutch mechanism (not shown) so that the indexing wheel 23 will rotate together with the gearwheel 40 in only one direction of rotation, the gearwheel being free to rotate in the opposite direction relative to the indexing wheel 23.

The actuator has a similar construction to the actuator 3 of the first embodiment and comprises a lever arm 7 with the mouthpiece 5 and piercing heads 9 upstanding from opposite sides thereof. However, in this embodiment, the user does not directly pivot the actuator 3. Instead, a cam pin 41 protrudes from the side of the lever arm 7 adjacent to the remote end opposite the end pivotally mounted to the housing 2. The cam pin 41 is located in a cam track or groove 42 formed on the inside surface of a cap 43 pivotally attached to the side of the housing 2 at the same end but spaced from the location at which the actuator 3 is pivotally attached to the housing 2. The cap 43 also carries a toothed gearwheel 44 attached thereto for rotation together with the cap 43, which lies in meshing engagement with the gearwheel 40 on the indexing wheel 23.

As has already been mentioned with reference to the first embodiment, the inhalation device according to the second embodiment also has a very simple construction with relatively few components. For example, if the gearwheel 44 is integrally formed together with the cap and the actuator 3 is formed together with the mouthpiece 5 and the piercing heads 9, the whole device can be formed from as few as 4, 5 or 6 moulded plastic parts.

Due to the small number of parts and simplicity of the device, there is more storage room within the device for blisters thereby reducing the frequency that it must be re-filled or replaced. It is intended that the devices of the present invention will have a capacity to hold between 1 and more than 100 doses although preferably it will be capable of holding between 1 and 60 doses and most preferably between 30 and 60 doses. The payload of each blister may be between 1 μg and 100 mg. However, preferably, the payload is in the region of 1 mg to 50 mg and most preferably between 10 mg and 20 mg. It will also be apparent that due to its simplicity, the device may be disposable once all the blisters contained therein have been used up. In this case, the housing may be formed as a permanently sealed enclosure to prevent tampering.

Operation of the inhaler according to the second embodiment will now be described with particular reference to FIGS. 12A and 12B. As can be seen in FIG. 12A, when the cap 43 is closed, the piercing heads 9 on the actuator 3 are held clear from the aperture 8 in the housing 2 by means of the cam pin 41 located in the cam track 42 in the cap 43. The cam track 42 is preferably shaped so that the cap 43 can be initially pivoted relative to the housing 2 by at least 90 degrees without any movement of the actuator 3 occurring thereby allowing inspection or cleaning of the mouthpiece 5 without piercing of a blister 6a. However, when the cap 43 is rotated relative to the housing 2 beyond 90 degrees, the cam pin 41 is guided by the track 42 causing the actuator 3 to pivot into a position shown in FIG. 12B in which the piercing elements 9 extend through the aperture 8 in the housing 2 and penetrate a blister 6b situated immediately behind the aperture 8 within the housing 2. At this stage, the dose may be inhaled through the mouthpiece 5.

As the cap 43 opens the gearwheel 40 rotates due to engagement with the gearwheel 44 on the cap 43. However, because of the one-way clutch mechanism, the indexing wheel 23 does not rotate as the cap 43 is opened and the gearwheel 40 is rotated in this first direction. However, once the cap 43 is rotated in the opposite direction, i.e. from the open to the closed position following inhalation, drive of the gearwheel 40 is transferred to the indexing wheel 23 so that it rotates and moves the next blister 6a into alignment with the aperture 8. It will be appreciated that during initial movement of the cap 43 from its open to its closed position, the actuator 3 will first be pivoted, due to the engagement of the cam pin 41 in the cam track 42, so that the piercing elements 9 are lifted out of the aperture 8 and back into the position shown in FIG. 12A.

It is envisaged that, in either embodiment, an opening or window could be provided in the housing 2 and a dose number printed on each blister 6a readable through the opening or window so that the user can monitor the number of doses that have been used or that remain in the device. This avoids the need for a complicated dose counting mechanism often found in conventional devices. Alternatively, the housing 2 could be wholly or partially formed from a transparent material so that the number of blisters 6 remaining in the device can clearly be seen through the walls of the housing 2.

As shown in the FIG. 13, the blister strip 6 provided for use with the inhaler 1 of the invention may be provided with serrations, cut-lines 50 or other frangible features to facilitate the separation of the blisters 6a from each other. Alternatively, or in addition to the frangible features, the edge of the blister strip 6 may be provided with notches 51 between each blister 6a to make the strip easier to tear.

Another embodiment of the device will now be described with reference to FIG. 14A to 19. This version of the device has the particular benefit of being small in size relative to the number of blisters that it may contain. Instead of placing the indexing wheel in its own cavity adjacent to the aperture in the housing through which the piercing heads extend, the indexing wheel is formed integrally with the hinge, which pivotally connects the actuating lever to the housing. This frees up more space within the housing for blister storage. As can be seen from the drawings, the device is able to contain a coil of at least 60 blisters.

Referring first to FIGS. 14A and 14B, there is shown two perspective views of the inhaler according to this embodiment. The inhaler 50 is similar to the inhaler 1 of the first embodiment and comprises a housing 51 having an actuator 52 in the form of a lever arm 53 pivotally mounted to the housing 51 at one end. A piercing member comprises a pair of piercing heads 54 that extend from the lever arm 53 and locate in an aperture 55 in the housing when the actuator 52 is in a closed position with the lever arm 53 lying substantially against the housing 51, as shown in FIG. 14A. A cap 56 is pivotally attached to the housing 51 and is operable to cover the mouthpiece 57 when the inhaler is not in use.

As with the first and second embodiments, the mouthpiece 57 is integral with the lever arm 53 although it has a triangular or semicircular section against which the lips can be placed, as opposed to a tubular section which is placed in the mouth. The shape of the mouthpiece and the airway construction within it is illustrated in the cross-sectional view of FIG. 18. It will be appreciated that the airway construction is very similar to the construction of the airway described with reference to the first and second embodiments and so no further description of it will be made here. However, it will be appreciated that because the indexing wheel is now located away from the region where the blister is pierced, the blister to be pierced is now supported in a blister support block 58 (see FIG. 17).

The device 50 includes an indexing wheel (not shown) incorporating a ratchet mechanism as has already been described with reference to the first and second embodiments, except that in this embodiment the indexing wheel has been made integral with the hinge about which the lever arm 53 pivots so that it rotates about the same axis as the lever arm 53.

When the cap 56 has been opened and the lever is pivoted from its closed position (as shown in FIG. 14A) into its open position (as shown in FIG. 14B), the indexing wheel rotates together with the lever due to engagement between a ratchet mechanism between the indexing wheel and the lever 53 and so draws a blister into alignment with the aperture 55 and locates in the blister support block 58. However, when the lever is returned to its closed position, the indexing wheel does not rotate due to the ratchet mechanism so the blister strip remains stationary. A second ratchet connection between the indexing wheel and the housing prevents backwards rotation the indexing wheel. During the final part of the return stroke, the piercing elements 54 extend through the aperture 55 and pierce the lid of the aligned blister. The dose is now ready for inhalation through the mouthpiece 57.

As described with reference to the previous embodiments, the device may incorporate a chamber to receive used blisters. However, this is not essential and the used blisters may simply be fed out of the device. A cutting edge 59 (see FIG. 16) may extend from the aperture against which used blisters may be torn off by pulling them against the edge in the direction indicated by the arrow in the drawing. The cutting edge may be serrated to facilitate detachment. It will be noted that the strip is prevented from being pulled out of the device by the piercing heads, which are located in a blister, and secures it in position.

It will be appreciated that any configuration of piercing member may be used including solid or hollow pins as well as piercing blades. However, it is desirable to include features that enhance the flow of air into the blister to aid entrainment and deagglomeration by, for example, introducing a swirling airflow into the blister. One particular arrangement of piercing head 60 which may be employed with any embodiment of the invention and which allows a freer flow of air into the blister will now be described with reference to FIGS. 18 and 19.

As can be seen from FIG. 18, the piercing member 60 is preferably integral with the lever arm that has a pair of apertures 61 therein for the flow of air into the blister and the flow of air together with the dose out of the blister. The piercing member 60 comprises a pair of piercing heads each of which comprises a pair of secondary cutting elements 62 spaced from each other and extending in a lateral direction from a pointed primary cutting element 63 which is mounted on and extends between the secondary cutting elements 62. The primary and secondary cutting elements 62,63 extend over one of the apertures 61 in the lever arm 53. Each of the secondary cutting elements 62 divided into first and second cutting members 62a, 62b that extend laterally from opposite sides of the primary cutting element 63. The first and second cutting members 62a, 62b are upwardly angled away from the lever arm and the primary cutting element upstands from the secondary cutting member 62 at the point where the first and second cutting members 62a,62b of each secondary cutting element 62 meet. The secondary cutting elements 62 incline inwardly toward each other so that the central piercing member 63 has diamond shape in side profile. As shown in FIG. 19B, this open construction allows more air to flow around the sides of the blister in comparison with the piercing member arrangement of FIG. 8A, as the side teeth restrict airflow into the blister (as shown in FIG. 19A).

It will be appreciated that the dimensions of the piercer of the present invention can be chosen to suit different sizes and shapes of blisters. Furthermore the number and arrangement of piercers can be varied within the scope of the invention. For example, a large blister may have a pair of larger piercers, or multiple pairs of smaller piercers, for example two piercers for the air inlet and two for the air outlet.

It will be further appreciated that the use of the piercer of this invention is not limited to the inhalers described in the embodiments and may be used with any inhaler comprising a puncturable blister.

Referring to FIGS. 20 to 26, there is shown another embodiment of the invention that will now be described in detail.

The inhaler 70, according to this embodiment, comprises a housing 71 having an actuator 72 pivotally mounted thereto for rotation relative to the housing 71 about an axis indicated by the line marked “A” in FIGS. 20 to 22. A cap 73 is pivotally attached to the housing 71 and may be moved between an open position, as shown in FIG. 20, and a closed position in which the cap 73 covers a mouthpiece 74 to protect it and to prevent the ingress of dirt into the housing 71 through the mouthpiece 74.

In FIG. 21, the actuator 72 has been pivoted about axis “A” from its closed position shown in FIG. 20 into its fully open position to reveal a piercing member, comprising a pair of piercing heads 75, upstanding from the actuator 72 and an aperture 76 in the housing 71 through which the piercing heads 75 extends when the actuator 72 is in its closed position. A finger grip 77 is integrally moulded into the front lip of the actuator 72 to facilitate movement of the actuator 72 by the user between its open and closed positions.

As with the previous embodiments, the housing 71 contains a coiled strip of blisters 78 (see FIG. 23) and one such blister 78a (see FIG. 21) is located in a piercing position in which it is visible through the aperture 76. It will be noted that each of the blisters in the strip 78 are numbered and the number of the blister located in a piercing position is also visible through the aperture 76. One edge of the aperture 76 is provided with a cutout 79 (see FIG. 21) to enable the number of this blister 78a to be seen by the user when the actuator 72 is in its open position.

As has already been described with reference to the embodiment of FIG. 4, a cover 80 is pivotally attached to the housing 71 and encloses a space to receive used blisters 78b that are fed into this space through a slot 81 (see FIG. 23) formed in the wall of the housing 71. It will be appreciated that the space enclosed by the cover 80 is sufficiently large enough to accommodate only a few used blisters 78b at a time and so a section of used blisters 78b must periodically be removed from those unused blisters 78 that remain in the housing 71. In this embodiment, as shown in FIG. 22, the cover 80 is pivotally hinged to the housing 71 for rotation about an axis which is substantially parallel to the direction of movement of used blisters 78b out of the housing 71. Even when the cover 80 is closed, there is a gap (not shown) between the cover 80 and the housing 71 so that, if a user does not remove a strip of used blisters 78b when the space is full, the used blisters 78b will pass through this gap and protrude out of the housing 71.

As can be seen from FIGS. 23 and 25, the housing 71 is preferably formed in two halves which, as with all the embodiments, may be formed from a translucent plastic such as polypropylene and which are held together using suitably positioned and integrally moulded clip-in mounting pins (not shown) that cooperate with corresponding mounting posts 83. In the side view of the device shown in FIG. 23, one half of the housing 71 has been removed so that the location and path of a coiled strip of blisters 78 through the housing 71 is clearly visible, as are the internal components of the device. The mouthpiece cap 73 and the cover 80 have been omitted from FIG. 23 for the purposes of clarity.

Although the two casing halves may be separable by the user to enable them to refill the housing with a fresh strip of blisters, it is also envisaged that the inhaler could be of the “single use” type in which a strip of blisters is located in the housing during assembly, which is then subsequently sealed. Once that strip of blisters has been exhausted, the whole device is simply thrown away. It will be appreciated that the simplicity of the preferred embodiments of the device and the fact that they are made from a relatively small number of components (no more than nine), all of which are made from a plastics material, means that it is very cheap to manufacture and so rendering it disposable after a single strip of blisters has been exhausted is a viable proposition. Sealing the housing during manufacture also renders the device tamperproof.

The blister strip 78 passes over a blister strip locator chassis 84 received in the housing 71 and mounted adjacent to the aperture 76. As can be most clearly seen from the exploded view of FIG. 25, the chassis 84 comprises two arcuately shaped parallel wall members 84a, 84b joined to and spaced from each other by a width which is only slightly greater than the width of the blister strip 78 so that the strip 78 (only a short section of which is shown in FIG. 25) passes between the wall members 84a, 84b and is guided and supported by them and by the upper wall of the housing 71 as the strip 78 passes therethrough. Each wall member 84a, 84b is provided with integrally moulded lugs 85 that locate between corresponding lugs 86 integrally moulded into the housing 71. Similarly, each wall member 84a, 84b has slots 87 which mate with corresponding locating features 82 on the housing 71 to firmly mount the strip locator chassis 84 in position.

The strip locator chassis 84 includes a resiliently deformable arm 88 depending from between the wall members 84a, 84b. The arm 88 is preferably integrally moulded together with the strip locator chassis 84 from a plastic material such as acetal. The free end of the arm 88 is divided into two forks 89 between which an indexing wheel 90 is rotatably mounted.

Referring now to FIG. 26, the indexing wheel 90 has four spokes 91 arranged in an “X” shape and it is positioned substantially coaxial with the axis “A” about which the actuator 72 rotates with respect to the housing 71. The housing 71 is also provided with indexing wheel anti-rotation and location ramps 92,93 which the indexing wheel 90 interacts with to selectively prevent and permit rotation of the indexing wheel 90, as will be explained in more detail later.

The actuator 72 includes a pair of flanges 94a,94b. One flange 94a has a shaped opening 95 that locates directly on a correspondingly shaped spigot 96 integrally formed on one-half of the housing 71. The other flange 94b is provided with a larger opening 97 that is shaped to receive a coupling plate 98 therein. The flange 94b is provided with a recess 99 in the edge of the opening 97 in which is received a locating tab 100 protruding from the coupling plate 98. The coupling plate 98 has a shaped opening 98a that locates on a correspondingly shaped spigot 101 on the other half of the housing 71. An arcuately shaped opening 105 in the housing 71 surrounds the spigot 101 through which extends an angularly shaped drive tooth 102, which protrudes inwardly from the coupling plate 98. The drive tooth 102 extends into a space between two spokes 91 of the indexing wheel 90 and its function will now be described with reference to FIG. 26.

FIG. 26 illustrates a series of drawings to show how the indexing mechanism works when the actuator 72 is rotated between its closed and open position and back to its closed position once again. The blister strip 78 has been omitted from FIG. 26 for clarity although it will be apparent that, as the indexing wheel 90 rotates, a blister will be located between a pair of spokes 91 and pulled through the housing 71.

Referring to FIG. 26A, the actuator 71 is in its closed position and the arm 88, with the indexing wheel mounted thereto, lies in an unstressed or relaxed state in which no external forces are applied to it. The drive tooth 102 can be seen positioned between two of the spokes 91a, 91b and spoke 91d is positioned between the anti-rotation and location ramps 92,93. The anti-rotation ramp 92 prevents any rotation of the indexing wheel 90 in a clockwise direction as viewed in the drawing.

When the actuator 71 is rotated towards its open position, in the direction of arrow “A” in FIG. 26B, the drive tooth 102 contacts spoke 91b. Further rotation of the actuator 71, as shown in FIG. 26C, causes the indexing wheel 90 to rotate, in an anti-clockwise direction as viewed in the drawing, due to the engagement between the drive tooth 102 and the spoke 91b, thereby indexing the blister strip 78.

As the indexing wheel 90 rotates, spoke 91c comes into contact with the anti-rotation ramp 92. When the anti-rotation ramp 92 and the spoke 91c engage, further rotation of the actuator 71 in the direction of arrow marked “A” causes the arm 88 to resiliently deform and deflect in an upward direction (in the direction of the arrow marked “B” in FIG. 26C) so that the spoke 91c can clear the anti-rotation ramp 92. When the actuator 71 has been rotated into its fully open position, the indexing wheel 90 has rotated through a full 90 degrees and spoke 91c clears the anti-rotation ramp 92 thereby allowing the indexing wheel 90 to drop back down and the arm 88 to return to its original undeformed state.

The actuator 71 is now rotated back into its closed position, in the direction of arrow “C” in FIG. 26E. The drive tooth 102 is shaped so that, on the return stroke of the actuator 71, it slides over the top of the preceding spoke 91a and does not rotate the indexing wheel 90 in a clockwise direction. As shown in FIG. 26E, engagement of the drive tooth 102 with the indexing wheel 90 actually causes the arm 88 and the indexing wheel 90 to deflect downwardly in the direction of arrow marked “D” in FIG. 26E. In this position, spoke 91c is pushed down in between the anti-rotation and location ramps 92,93 thereby preventing any rotation of the indexing wheel 90 in either direction.

At the completion of the return stroke, the piercing heads 75 pierce a previously unused blister that has just been indexed into place and is visible through the aperture 76 in the housing 71.

It will be appreciated that, if the actuator 71 is returned to is closed position before the full stroke is completed, the tooth 102 will engage the spoke 91a and cause the indexing wheel 90 to rotate in a clockwise direction back into its original position. This ensures that a partial index cannot take place and so the piercing heads 75 will always enter a blister.

Although the piercing heads 75 may be integrally formed together with the actuator 71, it is also envisaged that the piercing member may be formed as a separately moulded component 105, as shown in FIGS. 27, 27A and 27B, which locates in a walled recess 103 in the actuator 72, as shown in FIG. 28. The piercing heads then extend from this separately moulded component. This will now be described in more detail.

The piercing member 105 may be used with any of the embodiments of the inhalation device described herein and, as shown in FIGS. 27, 27A and 27B, comprises a main body portion 106 having an upper surface 107 which lies flush against the upper surface of a lid of a pierced blister 119 when the piercer has fully entered the blister 119. The piercing heads comprise one piercing tooth 108 upstanding from the upper surface 107 and another piercing tooth 109 upstanding from a relieved or recessed region 107a of the upper surface 107. The geometry of teeth 108,109 is similar to the geometry of the teeth already described with reference to FIGS. 18 and 19. Apertures 110,111 are formed in the upper surface 107 and recessed region 107a beneath teeth 108,109 respectively.

As can be seen in FIGS. 27A and 27B, the angles of the piercer are chosen to facilitate effective and clean cutting of the foil without tearing the foil in an uncontrolled manner. The preferred ranges and values for these angles are given in the table below:

| | | Value of embodiment of |
|---|---|---|
| Angle | Preferred range | FIGS. 27, 27A, 27B |
| a | 15°-45° | 33° |
| b | 15°-45° | 34° |
| c | 5°-30° | 15° |
| d | 5°-30° | 16° |

It may be advantageous to form the primary cutting element 63 so that it is positioned asymmetrically with respect to the secondary cutting elements 62. The first and second cutting members 62a,62b of each secondary cutting element 62 each extend laterally from the primary piercing element by different distances such that the two flaps formed by a piercing head are not the same size, as can be seen in FIG. 27A. As shown in the drawing the piercing heads 108,109 are arranged so that smaller flaps are formed towards the ends of the blister's major axis where the depth of the blister is shallower, and longer flaps are formed towards the centre of the blister where the blister is deeper. The relative length of the first and second cutting members 62a,62b is defined by the ratio k:j in FIG. 27A. Preferably this ratio is between 1 and 2. In the embodiment of FIGS. 27, 27A and 27B the ratio is 1.2. By making the flaps unequal sizes, agglomerates of medicament are less likely to get trapped within the blister.

A short tubular section 112 depends from the other side of the main body portion 106 in the opposite direction to the tooth 108 and is in communication with the aperture 110. The outer surface of the tubular section 112 has axially extending spacer ridges 113 for reasons that will become apparent. A mounting pin 114 also depends from the main body portion 106 to facilitate attachment of the piercing member 105 to the actuator 72.

When a user inhales through the mouthpiece 74, air is sucked through aperture 111 and into the blister 119 via an opening in the lid 119a of the blister 119 created by tooth 109. Tooth 109 upstands from a recessed region of the main body portion 106 so that a gap is created between the blister lid 119a and the surface of the recessed region 107a to allow free and unrestricted flow of air into the blister 119 through the aperture 109. The drug 119c contained in the blister 119 is entrained in the airflow entering the blister 119 formed by tooth 109 and is carried out of the blister 119 through the opening cut by tooth 108 through the aperture 110 and tubular section 112 into the mouthpiece 74 from where it passes into the patient's airway. The upper surface 107, around tooth 108 is shaped to fit closely against the blister lid when the teeth 108,109 have entered the blister 119 to their fullest extent so that leakage of air into the exit airflow between the upper surface 107 and the blister lid 119a is minimised.

As already described with reference to FIG. 9, to reduce the overall pressure drop across the device and make it easier for the patient to inhale a dose, outside air is introduced into the exit airflow through a bypass conduit 118. In this embodiment, the piercing head 105 is mounted to the actuator 72 via the tubular section 112 that locates within the walled recess 103. The ridges 113 form an interference fit with the walled recess 103 but gaps or spaces between the ridges 113 form a bypass conduit 118 through which bypass air is drawn into the mouthpiece 74 together with the airflow passing through the blister 119. It will be appreciated that the bypass air does not pass through the blister 119 but enters the mouthpiece 74 separately. This reduces the overall resistance to inspiratory flow, making the device easier to use. As has been described with reference to the embodiment of FIG. 9, mixing of bypass air with air that has passed through the blister 119 also enables more efficient dispersion of drug in the inspired air. A mesh 115 (see FIG. 29) may also be moulded into the mouthpiece 74 through which all the inspired air passes so as to provide additional dispersion.

Holes 114 are provided in a region where the mouthpiece 74 joins the actuator 72 through which air is fed via the aperture 111 into the blister 119 and, via the bypass conduit 118 formed by the spaces between ridges 113, into the mouthpiece 74.

The airflow through a pierced blister 119 and into the mouthpiece 74 is illustrated schematically in FIG. 29. When a patient inhales through the mouthpiece 74, air is drawn from outside through the holes 114 between the mouthpiece 74 and the actuator 72 from where it flows into the blister 119 through the aperture 111, as indicated by arrow marked “F”. In addition to inlet airflow through the aperture 111, air is also drawn into the blister 119 through the space between the lid 119a of the blister 119 and the recessed surface 107a, as indicated by arrow marked “G”. In addition to airflow into the blister 119, air is also drawn through the bypass conduit 118 (in the direction of the arrow marked “H”) formed by the spaces between the ridges 113 of the tubular section 112 of the piercing head 105 and joins the exit airflow leaving the blister 119 through the aperture 110 in the piercing member 105, in the direction of arrow marked “I”. The dose is entrained in the exit airflow and this airflow from the blister 119 together with the air that has flowed into the mouthpiece 74 via the bypass conduit 118 passes through the mesh 115 and out of the device into the patient's airway, in the direction of arrows marked “J”.

This embodiment as described has nine moulded components. While this is significantly fewer than other devices with a similar number of doses it is possible to reduce the component count still further. The case halves can, for example, be moulded as a single moulding connected by a moulded-in hinge at the base of the components. In assembly the two halves would be folded together to form the housing. Similarly, the cap and blister door can be integrally moulded.

In addition, as has been described the piercing element can be moulded as part of the actuator. In this way the number of moulded components can be reduced to five or six.

A final embodiment of an inhaler according to the invention will now be described with reference to FIG. 30.

It will be appreciated that it is advantageous for used blisters to be ejected from the device as this results in a smaller and simpler construction. If the device is to retain used blisters, then a take-up spool is required onto which the used blister strip is wound. The obvious disadvantage of a take-up spool is that at all times during use of the device there is an empty space within it. When the device is first used, the take-up spool is empty, and at the end of its life, the feed spool is empty. Accordingly, the device must be made larger to accommodate the blister strip both before and after use.

In an alternative embodiment of the present invention, the inhalation device retains used blisters in a more compact arrangement in which there is no unused space. This is achieved by forming the blister strip into an endless loop and mounting the loop in the housing in a state in which it has been wrapped around itself, as shown in FIG. 30.

Referring to FIG. 30, it can be seen that the housing 120 contains two spaced parallel walls 121, 122 to define a pair of parallel spiral channels 123,124 therebetween. The inner end of the channels 123,124 open out into a central chamber region 125 in which is rotatably mounted a feed spool 126 and a feed sprocket 127. The blister strip 130 passes from one channel 123 to the other channel 124 through the chamber region 125 and extends around the feed spool 126 and the feed sprocket 127 in an “S” shaped configuration. The blister strip 130 also passes out of one channel 124 and is wrapped around an indexing wheel (shown generally by reference numeral 128 in FIG. 30) before passing back into the other channel 123. The connections at both ends in effect create a single endless channel for the blister strip 130.

The blister strip 130 may be conventionally formed before its ends are subsequently joined together. If the length of the strip 130 matches the combined length of the two channels 123,124, the strip 130 can be loaded into the channels 123,124 and located around the teeth (not shown) of the indexing wheel 128 and the inner sprocket 127, as well as being guided around the spool 126.

The indexing wheel 128 indexes the strip 130 via a mouthpiece/actuator arrangement, as has already been described above with reference to FIGS. 20 to 26, although other indexing mechanisms may also be used.

If suitable low friction materials are used, the inner spool 126 and sprocket 127 need not be driven other than by the strip 78 itself. For a long strip 78, or to ensure reliable operation, the spool 126 and sprocket 127 may be connected to the indexing wheel 128 by a simple drive train, belt or similar mechanism (not shown).

As the strip 130 is endless, with regularly spaced blisters, then the user will be able to index the strip 130 indefinitely. Including a blank section 129 in the strip 130 that has no blisters can provide a clear indication that all blisters have been used. This could conveniently be provided at the point where the ends of the strip 130 are joined together. When this blank section 129 of the strip reaches the indexing wheel 128, the strip 78 will no longer be indexed as the indexing wheel 128 rotates, clearly indicating that the strip 130 has been exhausted. In the drawing, the strip 130 is shown with the blank section 129 located just after the indexing wheel 128. This is the position it will be in before the device has been used for the first time.

Features of the dry powder composition are very important to the efficiency of the delivery of the active agent to the lung. Therefore, the composition must be formulated to ensure that the particles of active agent are efficiently extracted from the blister or capsule by the passive device and dispensed in a form that encourages deposition in the deep lung of the patient, so that the active agent can have its desired local or systemic effect.

For formulations to reach the deep lung or the blood stream via inhalation, the active agent in the formulation must be in the form of very fine particles, for example, having a mass median aerodynamic diameter (MMAD) of less than 10 μm. It is well established that particles having an MMAD of greater than 10 μm are likely to impact on the walls of the throat and generally do not reach the lung. Particles having an MMAD in the region of 5 to 2 μm will generally be deposited in the respiratory bronchioles whereas particles having an MMAD in the range of 3 to 0.05 μm are likely to be deposited in the alveoli and to be absorbed into the bloodstream.

Preferably, for delivery to the lower respiratory tract or deep lung, the MMAD of the active particles is not more than 10 μm, and preferably not more than 5 μm, more preferably not more than 3 μm, and may be less than 2 μm, less than 1.5 μm or less than 1 μm. Especially for deep lung or systemic delivery, the active particles may have a size of 0.1 to 3 μm or 0.1 to 2 μm.

Ideally, at least 90% by weight of the active particles in a dry powder formulation should have an aerodynamic diameter of not more than 10 μm, preferably not more than 5 μm, more preferably not more than 3 μm, not more than 2.5 μm, not more than 2.0 μm, not more than 1.5 μm, or even not more than 1.0 μm.

When dry powders are produced using conventional processes, the active particles will vary in size, and often this variation can be considerable. This can make it difficult to ensure that a high enough proportion of the active particles are of the appropriate size for administration to the correct site. It is therefore desirable to have a dry powder formulation wherein the size distribution of the active particles is as narrow as possible. For example, the geometric standard deviation of the active particle aerodynamic or volumetric size distribution (σg), is preferably not more than 2, more preferably not more than 1.8, not more than 1.6, not more than 1.5, not more than 1.4, or even not more than 1.2. This will improve dose efficiency and reproducibility.

Fine particles, that is, those with an MMAD of less than 10 μm and smaller, tend to be increasingly thermodynamically unstable as their surface area to volume ratio increases, which provides an increasing surface free energy with this decreasing particle size, and consequently increases the tendency of particles to agglomerate and the strength of the agglomerate. In the inhaler, agglomeration of fine particles and adherence of such particles to the walls of the inhaler are problems that result in the fine particles leaving the inhaler as large, stable agglomerates, or being unable to leave the inhaler and remaining adhered to the interior of the inhaler, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung.

In an attempt to improve this situation and to provide a consistent FPF and FPD, dry powder formulations often include additive material. The additive material is intended to control the cohesion between particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles return to the form of small individual particles which are capable of reaching the lower lung.

However, the optimum stability of agglomerates to provide efficient drug delivery will depend upon the nature of the turbulence created by the particular device used to deliver the powder. Given that passive devices tend to create less turbulence than active devices, a particularly attention needs to be paid to the stability of the agglomerates formed. They will need to be stable enough for the powder to exhibit good flow characteristics during processing and loading into the device, whilst being unstable enough to release the active particles of respirable size upon actuation.

Preferably, the additive material is an anti-adherent material and it will tend to reduce the cohesion between particles and will also prevent fine particles becoming attached to the inner surfaces of the inhaler device. Advantageously, the additive material is an anti-friction agent or glidant and will give better flow of the pharmaceutical composition in the inhaler. The additive materials used in this way may not necessarily be usually referred to as anti-adherents or anti-friction agents, but they will have the effect of decreasing the cohesion between the particles or improving the flow of the powder. The additive materials are often referred to as force control agents (FCAs) and they usually lead to better dose reproducibility and higher fine particle fractions.

Therefore, an FCA, as used herein, is an agent whose presence on the surface of a particle can modify the adhesive and cohesive surface forces experienced by that particle, in the presence of other particles. In general, its function is to reduce both the adhesive and cohesive forces.

Known additive materials usually consist of physiologically acceptable material, although the additive material may not always reach the lung.

Preferred additive materials for used in dry powder formulations include amino acids, peptides and polypeptides having a molecular weight of between 0.25 and 1000 kDa and derivatives thereof.

It is particularly advantageous for the FCA to comprise an amino acid. The FCA may comprise or consist of one or more of any of the following amino acids: leucine, isoleucine, lysine, valine, methionine, and phenylalanine. The FCA may be a salt or a derivative of an amino acid, for example aspartame or acesulfame K. Preferably, the FCA consists substantially of an amino acid, more preferably of leucine, advantageously L-leucine. The D- and DL-forms may also be used. The FCA may comprise Aerocine™, amino acid particles as disclosed in the earlier patent application published as WO 00/33811.

The FCA may comprise or consist of one or more water soluble substances. This helps absorption of the FCA by the body if it reaches the lower lung.

The FCA may comprise or consist of dipolar ions, which may be zwitterions. It is also advantageous for the FCA to comprise or consist of a spreading agent, to assist with the dispersal of the composition in the lungs. Suitable spreading agents include surfactants such as known lung surfactants (e.g. ALEC®) which comprise phospholipids, for example, mixtures of DPPC (dipalmitoyl phosphatidylcholine) and PG (phosphatidylglycerol). Other suitable surfactants include, for example, dipalmitoyl phosphatidylethanolamine (DPPE), dipalmitoyl phosphatidylinositol (DPPI).

The FCA may comprise or consist of a metal stearate, for example, zinc stearate, magnesium stearate, calcium stearate, sodium stearate or lithium stearate, or a derivative thereof, for example, sodium stearyl fumarate or sodium stearyl lactylate.

The FCA may comprise or consist of one or more surface active materials, in particular materials that are surface active in the solid state, which may be water soluble or water dispersible, for example lecithin, in particular soya lecithin, or substantially water insoluble, for example solid state fatty acids such as oleic acid, lauric acid, palmitic acid, stearic acid, erucic acid, behenic acid, or derivatives (such as esters and salts) thereof, such as glyceryl behenate. Specific examples of such surface active materials are phosphatidylcholines, phosphatidylethanolamines, phosphatidylglycerols and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the FCA may comprise or consist of cholesterol. Other useful FCAs are film-forming agents, fatty acids and their derivatives, as well as lipids and lipid-like materials.

Other possible FCAs include sodium benzoate, hydrogenated oils which are solid at room temperature, talc, titanium dioxide, aluminium dioxide, silicon dioxide and starch.

In some embodiments, a plurality of different FCAs can be used.

Dry powder formulations often include coarse carrier particles of excipient material mixed with fine particles of active material. In such compositions, rather than sticking to one another, the fine active particles tend to adhere to the surfaces of the coarse carrier particles whilst in the inhaler device, but are supposed to release and become dispersed upon actuation of the dispensing device and inhalation into the respiratory tract, to give a fine suspension.

The inclusion of coarse carrier particles is also very attractive where very small doses of active agent are dispensed. It is very difficult to accurately and reproducibly dispense very small quantities of powder and small variations in the amount of powder dispensed will mean large variations in the dose of active agent where the powder comprises mainly active particles. Therefore, the addition of a diluent, in the form of large excipient particles will make dosing more reproducible and accurate.

Carrier particles may comprise or consist of any acceptable excipient material or combination of materials and preferably the material(s) is (are) inert and physiologically acceptable. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously the carrier particles are of a polyol. In particular the carrier particles may be particles of crystalline sugar, for example mannitol, dextrose or lactose. Preferably, the carrier particles are of lactose.

According to one embodiment of the present invention, the carrier particles are relatively large, compared to the particles of active material. This means that substantially all (by weight) of the carrier particles have a diameter which lies between 20 μm and 1000 μm, or between 50 μm and 1000 μm. Preferably, the diameter of substantially all (by weight) of the carrier particles is less than 355 μm and lies between 20 μm and 250 μm. In one embodiment, the carrier particles have a MMAD of at least 90 μm.

Preferably, at least 90% by weight of the carrier particles have a diameter between from 60 μm to 180 μm. The relatively large diameter of the carrier particles improves the opportunity for other, smaller particles to become attached to the surfaces of the carrier particles and to provide good flow and entrainment characteristics and improved release of the active particles in the airways to increase deposition of the active particles in the lower lung.

When adding coarse carrier particles to a composition of fine active particles it is important to ensure that the fine particles detach from the surface of the large particles upon actuation of the delivery device. To do this, it is known to include in the composition additive materials of the nature discussed above, as disclosed in WO 96/23485.

A 3-component system wherein the dry powder composition includes the pharmaceutically active agent, an additive material and carrier particles is generally expected to work well in a passive device. The presence of the carrier particles makes the powder easier to entrain in the air flow and extract from the blister, capsule or other storage means. The inclusion of carrier particles means that the powder is less cohesive and exhibits better flowability, compared with a powder consisting entirely of smaller particles, for example all having a diameter of less than 10 μm.

Relatively large amounts of coarse carrier are required in order to have the desired effect on the powder properties because the majority of the fine or ultra-fine active particles need to adhere to the surfaces of the carrier particles, otherwise the cohesive nature of the active particles still dominates the powder and results in poor flowability. The surface area of the carrier particles available for the fine particles to adhere to decreases with increasing diameter of the carrier particles. However, the flow properties tend to become worse with decreasing diameter. Hence, there is a need to find a suitable balance in order to obtain a satisfactory carrier powder, especially when the powder is to be dispensed using a passive inhaler device which can struggle to efficiently and reproducibly dispense powders with poor flowability.

However, the combination of coarse carrier particles and fine active particles has disadvantages. It can only be effectively used with a relatively low (usually only up to 5%) drug content. As more fine particles are included, more and more of the fine particles fail to become attached to the coarse carrier particles and segregation of the powder formulation becomes a problem. This, in turn, can lead to unpredictable and inconsistent dosing. The powder also becomes more cohesive and difficult to handle.

Furthermore, the size of the carrier particles used in a dry powder formulation can be influential on segregation. Segregation can be a catastrophic problem in powder handling during manufacture and the filling of devices or device components (such as capsules or blisters) from which the powder is to be dispensed. Segregation tends to occur where ordered mixes cannot be made sufficiently stable. Ordered mixes occur where there is a significant disparity in powder particle size. Ordered mixes become unstable and prone to segregation when the relative level of the fine component increases beyond the quantity which can adhere to the larger component surface, and so becomes loose and tends to separate from the main blend. When this happens, the instability is actually exacerbated by the addition of anti-adherents/glidants such as FCAs.

Solutions to some of the problems discussed above are already known. For example, flow problems associated with larger amounts of fine material, such as up to from 5 to 20% by total weight of the formulation, may be overcome by use of a large fissured lactose as carrier particles, as discussed in earlier patent applications published as WO 01/78694, WO 01/78695 and WO 01/78696.

In another embodiment, the excipient or carrier particles included in the formulations according to the present invention are relatively small, having a median diameter of about 3 to about 40 μm, preferably about 5 to about 30 μm, more preferably about 5 to about 20 and most preferably about 5 to about 15 μm. Such fine carrier particles, if untreated with an additive are unable to provide suitable flow properties when incorporated in a powder formulation comprising fine or ultra-fine active particles, especially when the formulation is to be dispensed by a passive device. Indeed, previously, particles in these size ranges would not have been regarded as suitable for use as carrier particles, and instead would only have been added in small quantities as a fine component in combination with coarse carrier particles. Such fine components are known to increase the aerosolisation properties of formulations containing a drug and a larger carrier, typically with median diameter 40 μm to 100 μm or greater. However, the quantity of such a fine excipient may be increased and such fine excipient particles may act as carrier particles if these particles are treated with an additive or FCA, even in the absence of coarse carrier particles. Such treatment can bring about substantial changes in the powder characteristics of the fine excipient particles and the powders they are included in. Powder density is increased, even doubled, for example from 0.3 g/cc to over 0.5 g/cc. Other powder characteristics are changed, for example, the angle of repose is reduced and contact angle increased.

Treated fine carrier particles having a median diameter of 3 to 40 μm are advantageous as their relatively small size means that they have a reduced tendency to segregate from the drug component, even when they have been treated with an additive to reduce cohesion. This is because the size differential between the carrier and drug is relatively small compared to that in conventional formulations which include fine or ultra-fine active particles and much larger carrier particles. The surface area to volume ratio presented by the fine carrier particles is correspondingly greater than that of conventional large carrier particles. This higher surface area, allows the carrier to be successfully associated with higher levels of drug than for conventional larger carrier particles. This makes the use of treated fine carrier particles particularly attractive in powder compositions to be dispensed by passive devices.

The ratios in which the different materials are present in a 2-component system (active and additive) or in a 3-component system (active, additive and carrier) will, of course, depend on the inhaler device used, the nature of the active particles and the required dose. The carrier particles, whether coarse, fine or a combination of both) may be present in an amount of at least 50%, more preferably 70%, advantageously 90% and most preferably 95% based on the total weight of the powder (including the carrier, active and additive). The appropriate amount of additive material to be included will also depend upon the manner in which it is incorporated into the composition, which is discussed in greater detail below

The chemical and physical properties of the fine particles comprising the pharmaceutically active agent also have an effect on the delivery of the dry powder composition from a passive device. However, whilst it is desirable to engineer the active particles to optimise their delivery by passive devices, it is also highly desirable to be able to prepare the fine particles using simple methods and simple apparatus.

Different approaches to particle engineering, allowing one to control and refine the particle cohesion, so that ideal powder behaviour and performance can be achieved and this can be matched to the device to be used to dispense the powder.

The present invention seeks to optimise the preparation of particles of active agent used in the dry powder composition dispensed using a passive DPI. In particular, the active particles may be engineered to provide a particle make-up and morphology which will produce high FPF and FPD results.

According to a second aspect of the present invention, methods are provided for preparing dry powder compositions for inclusion in the drug delivery systems according to the first aspect of the present invention, i.e. for delivery using a passive dry powder inhaler device.

In one embodiment, the amount of (effective) additive included in a dry powder composition, and the size and shape of the active particles may be accurately controlled and engineered by preparing composite particles comprising active material and additive material by spray drying. Spray drying is a well-known and widely used technique for producing particles of material.

Conventional spray drying techniques may be improved so as to produce active particles with enhanced chemical and physical properties so that they perform better when dispensed from a passive DPI than particles formed using conventional spray drying techniques. Such improvements are described in detail in the earlier patent application published as WO 2005/025535.

In particular, it is disclosed that co-spray drying an active agent with an FCA under specific conditions can result in particles with excellent properties which perform extremely well when administered by a passive DPI for inhalation into the lung.

It has been found that manipulating or adjusting the spray drying process can result in the FCA being largely present on the surface of the particles. That is, the FCA is concentrated at the surface of the particles, rather than being homogeneously distributed throughout the particles. This clearly means that the FCA will be able to reduce the tendency of the particles to agglomerate. This will assist the formation of unstable agglomerates that are easily and consistently broken up upon actuation of a passive DPI.

Where the spray drying takes place under “standard” parameters and using conventional spray drying apparatus, it has been found that spray drying an active agent with an FCA can lead to non-spherical particle morphology. Spray dried particles of pure active material are generally spherical in shape. However, at low concentrations of FCA, the surfaces of the particles show dimples or depressions. As the amount of co-spray dried FCA is increased, these dimples become more extreme, with the particles eventually having a shrivelled or wrinkled surface. The particles may, in selected cases, even burst as an extreme result of “blowing”, a phenomenon whereby the particles form a shell or skin which inflates due to the evaporation of the solvent, creating a raised internal vapour pressure and then may collapse or burst.

Droplets produced by the 2-fluid nozzle in a conventional spray drying system are initially dried at a relatively high rate during spray drying. This creates a viscous layer of material around the exterior of the liquid droplet. As the drying continues, the viscous layer is firstly stretched (like a balloon) by the increased vapour pressure inside the viscous layer as the solvent evaporates. The solvent vapour diffuses through the growing viscous layer until it is exhausted and the viscous layer then collapses, resulting in the formation of craters in the surface or wrinkling of the particles. The net effect of the inflation, stretching of the skin and deflation is the creation of significant numbers of craters and wrinkles or folds on the particle surface, which consequently results in a relatively low density particle which occupies a greater volume than a smooth-surfaced particle.

This change in the surface morphology of these co-spray dried particles may contribute to reduced cohesion between the particles. It has been argued that increased particle surface roughness or rugosity, such as is caused by surface wrinkles or craters, results in reduced particle cohesion and adhesion by minimising the surface contact area between particles. This reduction in particle cohesion can lead to the formation of relatively unstable agglomerates, which is beneficial where the powder composition is to be dispensed using a passive DPI. It has also previously been speculated that this particle morphology may even help the particles to fly when they are expelled for the inhaler device.

Despite this speculation relating to the benefits of the irregular shapes of these particles, the inventors actually believe that the chemical nature of the particle surfaces may be even more influential on the performance of the particles in terms of FPF, ED, etc. In particular, it is thought that the presence of hydrophobic moieties on the surface of particles is thought to be more significant in reducing cohesion that the presence of craters or dimples.

Indeed, in some circumstances it may be advantageous not to produce severely dimpled or wrinkled particles, as these can yield low density powders, with very high voidage between particles. Such powders occupy a large volume relative to their mass as a consequence of this form, and can result in packaging problems, i.e., much larger blisters or capsules are required for a given mass of powder.

It has been discovered that the FPF and FPD of the dry powder composition is also affected by the means used to create the droplets which are spray dried. Different means of forming droplets can affect the size and size distribution of the droplets, as well as the velocity at which the droplets travel when formed and the gas flow around the droplets. In this regard, the velocity at which the droplets travel when formed and the gas (which is usually air) flow around the droplets can dramatically affect size, size distribution and shape of resulting dried particles.

This aspect of the spray drying process is therefore important in the inventors' attempts to engineer particles with chemical and physical properties that provide good performance which the particles are dispensed using passive DPIs for pulmonary administration.

It has been found that it may be advantageous to control the formation of the droplets in the spray drying process, so that droplets of a given size and of a narrow size distribution are formed. Furthermore, controlling the formation of the droplets can allow control of the air flow around the droplets which, in turn, can be used to control the drying of the droplets and, in particular, the rate of drying. Controlling the formation of the droplets may be achieved by using alternatives to the conventional 2-fluid nozzles, especially avoiding the use of high velocity air flows. The following discussion of the use of alternative droplet forming means can be used in combination with all of the foregoing factors which provide improvements in the performance of the spray dried particles, as will become clear.

According to another embodiment of the invention, the active agent is spray dried using a spray drier comprising a means for producing droplets moving at a controlled velocity and of a predetermined droplet size. The velocity of the droplets is preferably controlled relative to the body of gas into which they are sprayed. This can be achieved by controlling the droplets' initial velocity and/or the velocity of the body of gas into which they are sprayed, for example by using an ultrasonic nebuliser (USN) to produce the droplets.

One type of ultrasonic nebuliser which may be used in the present invention is described in the European patent application published as EP 0931595A1. This patent application describes ultrasonic nebulisers which work extremely well in putting the present invention into practice, despite the fact that the nebulisers are intended for use as air humidifiers. The droplets produced are of an ideal size range with a small size distribution for use in a spray drying process. What is more, the nebulisers have a very high output rate of several litres of feed liquid per hour and up to of the order of 60 litres per hour in some of the devices produced and sold by the company Areco. This is very high compared to the 2-fluid nozzles used in conventional spray drying apparatus and it allows the spray drying process to be carried out on a commercially viable scale. Other suitable ultrasonic nebulisers are disclosed in U.S. Pat. No. 6,051,257 and in WO 01/49263.

The gas speed around the droplet will affect the speed with which the droplet dries. In the case of droplets which are moving quickly, such as those formed using a 2-fluid nozzle arrangement (spraying into air), the air around the droplet is constantly being replaced. As the solvent evaporates from the droplet, the moisture enters the air around the droplet. If this moist air is constantly replaced by fresh, dry air, the rate of evaporation will be increased. In contrast, if the droplet is moving through the air slowly, the air around the droplet will not be replaced and the high humidity around the droplet will slow the rate of drying. The rate at which a droplet dries affects various properties of the particles formed, including FPF and FPD.

A further advantage of the use of USNs to produce droplets in the spray drying process is that the particles which are produced are small, spherical in shape and are dense. These particles surprisingly perform very well when dispensed using a passive DPI and provide improved dosing. It is thought that the size and shape of the particles produced reduce the drug's device retention to very low levels.

In addition, the USNs can produce very small droplets relative to other known atomiser types and this, in turn, leads to the production of very small particles. The particles produced by USNs tend to be within the size range of 0.5 to 5 μm, or even 0.5 to 3 μm. This compares very favourably with the particle sizes which tend to be obtained using conventional spray drying techniques and apparatus, or obtained by milling. Both of these latter methods produce particles with a minimum size of around 1 μm. These advantages associated with the use of USNs are discussed in greater detail below.

When viewed using scanning electron micrographs (SEMs), the shape of particles formed by co-spray drying an active agent and an additive (in this case leucine) using a USN was found to dramatically differ from that of particles formed using a conventional 2-fluid nozzle spray drying technique. The distinctive dimples or wrinkles are less evident when the particles are spray dried using a USN. Despite this, the co-spray dried particles formed using a USN still have an improved FPF and FPD over particles formed in the same way but without the FCA. In this case, this improvement is clearly not primarily due to the shape of the particles, nor is it due to any increase in density or rugosity.

It is believed that the concentration of additive at the surface of the solid particles contributes to the excellent FPF and FPD observed and this is governed by several factors. These include the concentration of the additive in the solution which forms the droplets, the relative solubility of additive compared to the active agent, the surface activity of the additive, the mass transport rate within the drying droplet and the speed at which the droplets dry. If drying is very rapid it is thought that the additive concentration at the particle's surface will be lower than that for a slower drying rate. The surface concentration of the additive is determined by the rate of its transport or migration to the surface, and its precipitation rate, during the drying process.

As the gas speed around droplets formed using a USN is low in comparison to that around droplets formed using conventional 2-fluid nozzles, droplets formed using a USN dry more slowly. The additive concentration on the shell of droplets and dried particles produced using a USN can be higher as a result. It is considered that these effects reduce the rate of solvent evaporation from the droplets and reduce “blowing” and, therefore, are responsible for the physically smaller and smoother primary particles we have observed.

It is also speculated that the slower drying rate which is expected when the droplets are formed using USNs allows the additive to migrate to the surface of the droplet during the drying process. This migration may be further assisted by the presence of a solvent which encourages the hydrophobic moieties of the additive to become positioned on the surface of the droplet. An aqueous solvent is thought to be of assistance in this regard.

With the FCA being able migrate to the surface of the droplet so that it is present on the surface of the resultant particle, it is clear that a greater proportion of the FCA which is included in the droplet will actually have the force controlling effect (as the FCA must be present on the surface in order for it to have this effect). Therefore, it also follows that the use of USNs has the further advantage that it requires the addition of less FCA to produce the same force controlling effect in the resultant particles, compared to particles produced using conventional spray drying methods.

Studies of the particles produced by spray drying using USNs have led to the discovery that the bulk density of ultra-fine drug powders can be beneficially increased whilst also improving aerosolisation characteristics, even when the particles are dispensed using a passive DPI. The key to improved aerosolisation in a denser particle is the presence of the additive in the surface of the spray dried particles, without which the benefits of densification cannot be realised.

Thus, powders according to some embodiments of the present invention may preferably have a tapped density of more than 0.1 g/cc, more than 0.2 g/cc, more than 0.3 g/cc, more than 0.4 g/cc, or more than 0.5 g/cc. The inclusion of such relatively dense particles of active material in dry powder compositions unexpectedly leads to good FPFs and FPDs when the compositions are dispensed using a passive DPI.

Similar results to those shown above when using USNs are expected for spray drying using other means which produce low velocity droplets at high output rates. For example, further alternative nozzles may be used, such as electrospray nozzles or vibrating orifice nozzles. These nozzles, like the ultrasonic nozzles, are momentum free, resulting in a spray which can be easily directed by a carrier air stream, however, their output rate is generally lower.

The spray drying processes described above may include a further step wherein the moisture content of the spray dried particles is adjusted to allow fine-tuning of some of the properties of the particles. The amount of moisture in the particles will affect various particle characteristics, such as density, porosity, flight characteristics, and the like.

In one embodiment, the moisture adjustment or profiling step involves the removal of moisture. Such a secondary drying step can involve freeze-drying, wherein the additional moisture is removed by sublimation, or vacuum drying. Alternatively, the moisture profiling involves increasing the moisture content of the spray dried particles. Preferably, the moisture is added by exposing the particles to a humid atmosphere. The amount of moisture added can be controlled by varying the humidity and/or the length of time for which the particles are exposed to this humidity.

According to another, alternative embodiment of the present invention, the preparation of particles of the dry powder composition is optimised for delivery using a passive DPI by engineering the particles using a bespoke milling processes.

In the conventional use of the word, “milling” means the use of any mechanical process which applies sufficient force to the particles of active material that it is capable of breaking coarse particles (for example, particles with a MMAD greater than 100 μm) down to fine particles (for example, having a MMAD not more than 50 μm). In the present invention, the term “milling” also refers to deagglomeration of particles in a formulation, with or without particle size reduction. The particles being milled may be large or fine prior to the milling step.

Co-milling or co-micronising particles of active agent and particles of additive will result in the additive material becoming deformed and being smeared over or fused to the surfaces of fine active particles. These resultant composite active particles have been found to be less cohesive after the milling treatment. If a significant reduction in particle size is also required, co-jet milling is preferred, as disclosed in the earlier patent application published as WO 2005/025536. The co-jet milling process can result in composite active particles with low micron or sub-micron diameter, and these particles exhibit particularly good FPF and FPD, even when dispensed using a passive device.

The co-jet milling may, in certain circumstances, be more efficient in the presence of the additive material than it is in the absence of the additive material. The benefits are that it is therefore possible to produce smaller particles for the same mill, and it is possible to produce milled particles with less energy. Co-jet milling should also reduce the problem of amorphous content by both creating less amorphous material, as well as hiding it below a layer of additive material. The impact forces of the co-jet milling are sufficient to break up agglomerates of drug, even micronised drug, and are effective at distributing the additive material to the consequently exposed faces of the particles.

Different grinding and injection pressures may be used in order to produce particles with different coating characteristics which affect the performance of the powder compositions including these co-jet milled particles in passive inhaler devices.

Co-jet milling may be carried out at grinding pressures between 0.1 and 12 bar. Varying the pressure allows one to control the degree of particle size reduction. At pressures in the region of 0.1-3 bar, more preferably 0.5-2 bar and most preferably 1-2 bar, the co-jet milling will primarily result in blending of the active and additive particles, so that the additive material adheres to and coats the active particles. When the co-jet milling is carried out at such relatively low pressures, the resultant particles have been shown to perform well when dispensed using passive devices. It is speculated that this is because the particles are larger than those produced by co-jet milling at higher pressures and these relatively larger particles are more easily extracted from the blister, capsule or other storage means in the passive device, due to less cohesion and better flowability.

Where co-jet milling is carried out at a grinding pressure of between 3 and 12 bar, this results in a reduction of the sizes of the active and additive particles. However, the extremely small composite active particles (having an MMAD of between 3 and 0.5 μm) tend to exhibit relatively poor FPFs and FPDs when dispensed using a passive inhaler device, as powder formulations comprising such fine particles exhibit high cohesiveness.

The co-milling processes according to the present invention can also be carried out in two or more stages, to combine the beneficial effects of the milling at different pressures and/or different types of milling or blending processes. The use of multiple steps allows one to tailor the properties of the co-jet milled particles to suit a particular inhaler device, a particular drug and/or to target particular parts of the lung.

In one embodiment, the milling process is a two-step process comprising first milling the drug on its own to obtain the (very) small particle sizes possible using this type of milling. In one embodiment, this milling step involves jet milling, preferably at high grinding pressures. Next, the milled drug is co-milled with an additive material. Preferably, this second step results in the coating of the small active particles with the additive material. In one embodiment, this second step involves jet milling, preferably at lower grinding pressures.

The additive material may also be milled on its own prior to the co-milling step. This milling may be conducted in a jet mill, a ball mill, a high pressure homogeniser or alternative known ultrafine milling methods. The particles of additive material are preferably in a form with 90% of the particles by mass of diameter <10 μm, more preferably <5 μm, more preferably <2 μm, more preferably <1 μm and most preferably <0.5 μm,

This two-step process produces better results than simply co-jet milling the active material and additive material at a high grinding pressure. Experimental results discussed below show that the two-step process results in smaller particles and less throat deposition than simple co-jet milling of the materials at a high grinding pressure.

In another embodiment of the present invention, the particles produced using the two-step process discussed above subsequently undergo mechanofusion or an equivalent compressive process. This final mechanofusion step is thought to “polish” the composite active particles, further rubbing the additive material into the particles. This allows one to enjoy the beneficial properties afforded to particles by mechanofusion, in combination with the very small particles sizes made possible by the co-jet milling.

According to a further embodiment of the present invention, a powder composition is provided which is prepared by a method comprising co-milling active particles with an additive material, separately co-milling carrier particles with an additive material, and then combining the co-milled active and carrier particles.

The co-milling steps preferably produce composite particles of active and additive material or carrier and additive material.

The powder formulations prepared according to these methods exhibit excellent powder properties that may be tailored to the active agent and to the dispensing device to be used, as well as to various other factors. In particular, the co-milling of active and carrier particles in separate steps allows different types of additive material and different quantities of additive material to be milled with the active and carrier particles. Consequently, the additive material can be selected to match its desired function, and the minimum amount of additive material can be used to match the relative surface area of the particles to which it is being applied.

In one embodiment, the active particles and the carrier particles are both co-milled with the same additive material or additive materials. In an alternative embodiment, the active and carrier particles are co-milled with different additive materials.

In one embodiment of the invention, active particles of less than about 5 μm diameter are co-milled with an appropriate amount of an additive or force control agent, whilst carrier particles with a median diameter in the range of about 3 μm to about 40 μm are separately co-milled with an appropriate amount of an additive.

The additive material is preferably in the form of a coating on the surfaces of the active and carrier particles. The coating may be a discontinuous coating. In another embodiment, the additive material may be in the form of particles adhering to the surfaces of the active and carrier particles. Preferably, the additive material actually becomes fused to the surfaces of the active and carrier particles.

The co-milling or co-micronising of active and additive particles may involve compressive type processes, such as mechanofusion, cyclomixing and related methods such as those involving the use of a Hybridiser or the Nobilta. The principles behind these processes are distinct from those of alternative milling techniques in that they involve a particular interaction between an inner element and a vessel wall, and in that they are based on providing energy by a controlled and substantial compressive force, preferably compression within a gap of predetermined width.

For example, fine active particles and additive particles are fed into the Mechanofusion driven vessel (such as a Mechanofusion system (Hosokawa Micron Ltd)), where they are subject to a centrifugal force which presses them against the vessel inner wall. The inner wall and a curved inner element together form a gap or nip in which the particles are pressed together. The powder is compressed between the fixed clearance of the drum wall and a curved inner element with high relative speed between drum and element. As a result, the particles experience very high shear forces and very strong compressive stresses as they are trapped between the inner drum wall and the inner element (which has a greater curvature than the inner drum wall). The particles are pressed against each other with enough energy to locally heat and soften, break, distort, flatten and wrap the additive particles around the active particles to form coatings. The energy is generally sufficient to break up agglomerates and some degree of size reduction of both components may occur. Whilst the coating may not be complete, the deagglomeration of the particles during the process ensures that the coating may be substantially complete, covering the majority of the surfaces of the particles.

The milling processes apply a high enough degree of force to break up tightly bound agglomerates of fine or ultra-fine particles, such that effective mixing and effective application of the additive material to the surfaces of those particles is achieved.

Ball milling is a milling method used in many of the prior art co-milling processes. Centrifugal and planetary ball milling are especially preferred.

Jet mills are capable of reducing solids to particle sizes in the low-micron to submicron range. The grinding energy is created by gas streams from horizontal grinding air nozzles. Particles in the fluidised bed created by the gas streams are accelerated towards the centre of the mill, colliding with slower moving particles. The gas streams and the particles carried in them create, a violent turbulence and, as the particles collide with one another, they are pulverized.

High pressure homogenisers involve a fluid containing the particles being forced through a valve at high pressure, producing conditions of high shear and turbulence. Suitable homogenisers include EmulsiFlex high pressure homogenisers which are capable of pressures up to 4000 bar, Niro Soavi high pressure homogenisers (capable of pressures up to 2000 bar) and Microfluidics Microfluidisers (maximum pressure 2750 bar).

Milling may, alternatively, involve a high energy media mill or an agitator bead mill, for example, the Netzsch high energy media mill, or the DYNO-mill (Willy A. Bachofen AG, Switzerland).

All of these processes create high-energy impacts between media and particles or between particles. In practice, while these processes are good at making very small particles, it has been found that the ball mill, jet mill and the homogenizer may not be as effective in producing dispersion improvements in resultant drug powders as the compressive type processes. It is believed that the impact processes discussed above are not as effective in producing a coating of additive material on each particle as the compressive type processes.

An especially desirable aspect of the co-milling processes is that the additive material becomes deformed during the milling and may be smeared over or fused to the surfaces of the active particles. However, in practice, this compression process produces little or no size reduction of the drug particles, especially where they are already in a micronised form (i.e. <10 μm). The only physical change which may be observed is a plastic deformation of the particles to a rounder shape.

For the purposes of this invention, all forms of co-milling and co-micronisation are encompassed, including methods that are similar or related to all of those methods described above. For example, methods similar to Mechanofusion are encompassed, such as those utilizing one or more very high-speed rotors (i.e. 2000 to 50000 rpm) with blades or other elements sweeping the internal surfaces of the vessels with small gaps between wall and blade (i.e. 0.1 mm to 20 mm). Conventional methods comprising co-milling active material with additive materials (as described in WO 02/43701) are also encompassed. These methods result in composite active particles comprising ultra-fine active particles and/or carrier particles with an amount of the additive material on their surfaces.

Thus, the milling methods used in the present invention are simple and cheap compared to the complex previous attempts to engineer particles, providing practical as well as cost benefits. A further benefit associated with the present invention is that the powder processing steps do not have to involve organic solvents. Such organic solvents are common to many of the known approaches to powder processing and are known to be undesirable for a variety of reasons.

The milling processes can be specifically selected for the different steps and for the different active, additive and carrier materials and particles. For example, the active particles may be co-jet milled or homogenized with the additive, whilst the carrier particles may be mechanofused with the additive. The co-milling processes according to the present invention may be carried out in two or more stages, to provide beneficial effects. Various combinations of types of co-milling and/or additive material may be used, in order to obtain advantages. Within each step, multiple combinations of co-milling and other processing steps may be used. For example, milling at different pressures and/or different types of milling or blending processes may be combined, to tailor the properties of the milled particles to suit a particular inhaler device, a particular drug and/or to target particular parts of the lung.

The benefits of the methods according to the present invention are illustrated by the experimental data set out below.

### EXAMPLE 1

### Mechanofused Budesonide with Magnesium Stearate

This example studied magnesium stearate (MgSt) processed with budesonide. The blends were prepared by Mechanofusion using the Hosokawa AMS-MINI, with blending being carried out for 60 minutes at approximately 4000 rpm.

The magnesium stearate used was a standard grade supplied by Avocado Research Chemicals Ltd. The drug used was micronised budesonide. The powder properties were tested using the Miat Monohaler™.

Blends of budesonide and magnesium stearate were prepared at different weight percentages of magnesium stearate. Blends of 5% w/w and 10% w/w, were prepared and then tested. Tests using a multi stage liquid impinger (MSLI) and a twin stage impinger (TSI) were carried out on the blends. The results, which are summarised below, indicate a high aerosolisation efficiency. However, this powder had poor flow properties, and was not easily handled, giving high device retention.

| | | FPD | ED | |
|---|---|---|---|---|
| Formulation | FPF(ED) | (mg) | (mg) | Method |
| Budesonide:magnesium | 73% | 1.32 | 1.84 | MSLI |
| stearate (5% w/w) | | | | |
| Budesonide:magnesium | 80% | 1.30 | 1.63 | TSI |
| stearate (10% w/w) | | | | |

### EXAMPLE 2

### Mechanofused Budesonide with Fine Lactose and MgSt

A further study was conducted to look at the Mechanofusion of a drug with both a force control agent and fine lactose particles. The additive or force control agent used was magnesium stearate (Avocado) and the fine lactose was Sorbolac 400 (Meggle). The drug used was micronised budesonide.

The blends were prepared by Mechanofusion of all three components together using the Hosokawa AMS-MINI, blending was carried out for 60 minutes at approximately 4000 rpm.

Formulations were prepared using the following concentrations of budesonide, magnesium stearate and Sorbolac 400:
5% w/w budesonide, 6% w/w magnesium stearate, 89% w/w Sorbolac 400; and
20% w/w budesonide, 6% w/w magnesium stearate, 74% w/w Sorbolac 400.

TSIs and MSLIs were performed on the blends. The results, which are summarised below, indicate that, as the amount of budesonide in the blends increased, the FPF results increased. Device and capsule retention were notably low in these dispersion tests (<5%), however a relatively large level of magnesium stearate was used and this was applied over the entire composition.

| | FPF(ED) | FPF(ED) |
|---|---|---|
| Formulation | (TSI) | (MSLI) |
| 5:6:89 | 66.0% | 70.1% |
| 20:6:74 | 75.8% | — |

As an extension to this work, different blending methods of budesonide, magnesium stearate and Sorbolac 400 were investigated further. Two formulations were prepared in the Glen Creston Grindomix. This mixer is a conventional food-processor style bladed mixer, with 2 parallel blades.

The first of these formulations was a 5% w/w budesonide, 6% w/w magnesium stearate, 89% w/w Sorbolac 400 blend prepared by mixing all components together at 2000 rpm for 20 minutes. The formulation was tested by TSI and the results, when compared to those for the mechanofused blends, showed the Grindomix blend to give lower FPF results (see table below).

The second formulation was a blend of 90% w/w of mechanofused magnesium stearate:Sorbolac 400 (5:95) pre-blend and 10% w/w budesonide blended in the Grindomix for 20 minutes. The formulation was tested by TSI and MSLI.

It was also observed that this formulation had notably good flow properties for a material comprising such fine particles. This is believed to be associated with the Mechanofusion process.

| | FPF (ED) | FPF |
|---|---|---|
| Formulation | (TSI) | (MSLI) |
| Grindomix 5:6:89% | 57.7 | — |
| Grindomix 10% budesonide | 65.9 | 69.1 |
| (Mechanofused pre-blend) | | |

### EXAMPLE 3

### Mechanofused Salbutamol with Fine Lactose and MgSt

A further study was conducted to look at the Mechanofusion of an alternative drug with both a force control agent and fine lactose particles. The additive or force control agent used was magnesium stearate and the fine lactose was Sorbolac 400 (Meggle). The drug used was micronised salbutamol sulphate. The blends were prepared by Mechanofusion using the Hosokawa AMS-MINI, blending for 10 minutes at approximately 4000 rpm.

Formulations prepared were:
20% w/w salbutamol, 5% w/w magnesium stearate, 75% w/w Sorbolac 400; and
20% w/w salbutamol, 2% w/w magnesium stearate, 78% w/w Sorbolac 400.

NGIs were performed on the blends and the results are set out below. Device and capsule retention were again low in these dispersion tests (<10%).

| Formulation | FPF (ED) | FPF (ED) |
|---|---|---|
| 20:5:75 | 80% | 74% |
| 20:2:78 | 78% | 70% |

### EXAMPLE 4

### Preparation of Mechanofused Formulation for a Passive Device

20 g of a mix comprising 20% micronised clomipramine, 78% Sorbolac 400 (fine lactose) and 2% magnesium stearate were weighed into the Hosokawa AMS-MINI Mechanofusion system via a funnel attached to the largest port in the lid with the equipment running at 3.5%. The port was sealed and the cooling water switched on. The equipment was run at 20% for 5 minutes followed by 80% for 10 minutes. The equipment was switched off, dismantled and the resulting formulation recovered mechanically.

20 mg of the collected powder formulation was filled into size 3 capsules and fired from a Monohaler™ into an NGI. The FPF measured was good, being greater than 70%.

The data above suggest that magnesium stearate content in the region 5-20% yielded the greatest dispersibility. Above these levels, experience suggests significant sticking inside the device could occur, and the quantities used became unnecessary for further performance improvement.

Fine particle fraction values were consistently obtained in the range 50 to 60%, and doubled in comparison with controls containing no magnesium stearate.

### EXAMPLE 5

### Mechanofused Apomorphine and Mechanofused Fine Lactose

Firstly, 15 g of micronised apomorphine and 0.75 g leucine are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is then switched off, dismantled and the resulting formulation recovered mechanically.

Next, 19 g of Sorbolac 400 lactose and 1 g leucine are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically.

4.2 g of the apomorphine-based material and 15.8 g of the Sorbolac-based material are combined in a high shear mixer for 5 minutes, and the resulting powder is then passed through a 300 micron sieve to form the final formulation. 2 mg of the powder formulation are filled into blisters and fired from an Aspirair device into an NGI. An FPF of over 50% was obtained with MMAD 1.5 μm, illustrating this system gave a very good dispersion. The device retention was also very low, with only ˜1% left in the device and 7% in the blister.

### EXAMPLE 6

### Mechanofused Clomipramine and Mechanofused Fine Lactose

Firstly, 20 g of a mix comprising 95% micronised clomipramine and 5% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is then switched off, dismantled and the resulting formulation recovered mechanically.

Next, 20 g of a mix comprising 99% Sorbolac 400 lactose and 1% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically.

4 g of the clomipramine-based material and 16 g of the Sorbolac-based material are combined in a high shear mixer for 10 minutes, to form the final formulation. 20 mg of the powder formulation are filled into size 3 capsules and fired from a Monohaler™ into an NGI.

### EXAMPLE 7

### Mechanofused Theophylline and Mechanofused Fine Lactose

Firstly, 20 g of a mix comprising 95% micronised theophylline and 5% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is then switched off, dismantled and the resulting formulation recovered mechanically.

Next, 20 g of a mix comprising 99% Sorbolac 400 lactose and 1% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically.

4 g of the theophylline-based material and 16 g of the Sorbolac-based material are combined in a high shear mixer for 10 minutes, to form the final formulation. 20 mg of the powder formulation are filled into size 3 capsules and fired from a Monohaler™ into an NGI.

The active agent used in this example, theophylline, may be replaced by other phosphodiesterase inhibitors, including phosphodiesterase type 3, 4 or 5 inhibitors, as well as other non-specific ones.

### EXAMPLE 8

### Jet Milled Clomipramine and Mechanofused Fine Lactose

20 g of a mix comprising 95% micronised clomipramine and 5% magnesium stearate are co-jet milled in a Hosokawa AS50 jet mill.

20 g of a mix comprising 99% Sorbolac 400 (fine lactose) and 1% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically.

4 g of the clomipramine-based material and 16 g of the Sorbolac-based material are combined in a high shear mixer for 10 minutes, to form the final formulation.

20 mg of the powder formulation are filled into size 3 capsules and fired from a Monohaler™ into an NGI.

A number of micronised drugs were co-jet milled with magnesium stearate for the purposes of replacing the clomipramine in this example. These micronised drugs included budesonide, formoterol, salbutamol, heparin, insulin and clobazam. Further compounds are considered suitable, including the classes of active agents and the specific examples listed above.

### EXAMPLE 9

### Jet Milled Bronchodilator and Mechanofused Fine Lactose

20 g of a mix comprising 95% micronised bronchodilator drug and 5% magnesium stearate are co-jet milled in a Hosokawa AS50 jet mill.

20 g of a mix comprising 99% Sorbolac 400 lactose and 1% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically.

4 g of the drug based material and 16 g of the Sorbolac based material are combined in a high shear mixer for 10 minutes, to form the final formulation. 20 mg of the powder formulation is filled into size 3 capsules and fired from a Monohaler™ into an NGI.

The results of these experiments are expected to show that the powder formulations prepared using the method according to the present invention exhibit further improved properties such as FPD, FPF, as well as good flow and reduced device retention and throat deposition.

In accordance with the present invention, the % w/w of additive material will typically vary. Firstly, when the additive material is added to the drug, the amount used is preferably in the range of 0.1% to 50%, more preferably 1% to 20%, more preferably 2% to 10%, and most preferably 3 to 8%. Secondly, when the additive material is added to the carrier particles, the amount used is preferably in the range of 0.01% to 30%, more preferably of 0.1% to 10%, preferably 0.2% to 5%, and most preferably 0.5% to 2%. The amount of additive material preferably used in connection with the carrier particles will be heavily dependant upon the size and hence surface area of these particles.

The powders of the present invention are extremely flexible and therefore have a wide number of applications, for both local application of drugs in the lungs and for systemic delivery of drugs via the lungs. The present invention is also applicable to nasal delivery, and powder formulations intended for this alternative mode of administration to the nasal mucosa.

The size of the doses of active agent can vary from micrograms to tens of milligrams. The fact that dense particles may be used, in contrast to conventional thinking, means that larger doses can be administered without needing to administer large volumes of powder and the problems associated therewith.

The dry powder formulations may be pre-metered and kept in foil blisters which offer chemical and physical protection whilst not being detrimental to the overall performance. Indeed, the formulations thus packaged tend to be stable over long periods of time, which is very beneficial, especially from a commercial and economic point of view.

1. A drug delivery system for pulmonary administration of at least one pharmaceutically active agent or drug, the system comprising a passive dry powder inhaler device and a dry powder composition, wherein the powder composition comprises the at least one pharmaceutically active agent or drug and wherein the combination of the device and the composition ensure that at least 50% of the metered dose of the at least one active agent or drug is dispensed by the device upon actuation in a form that will allow deposition in the lung of a patient.

2. A drug delivery system as claimed in claim 1, wherein the metered dose is stored in the device in a capsule or blister.

3. A drug delivery system as claimed in claim 2, wherein the amount of active agent retained in the blister or capsule following actuation of the device is less than 15%.

4. A drug delivery system as claimed in claim 1, wherein the amount of the powder composition retained in the device following actuation is less than 15%.

5. A drug delivery system as claimed in claim 1, wherein, upon being expelled from the device, the powder composition has a dosing efficiency at 5 μm of at least 40%.

6. A drug delivery system as claimed in claim 1, wherein, upon being expelled from the device, the powder composition has a dosing efficiency at 3 μm of at least 30%.

7. A drug delivery system as claimed in claim 1, wherein, upon being expelled from the device, the powder composition has a dosing efficiency at 2 μm of at least 20%.

8. A drug delivery system as claimed in claim 1, wherein the amount of active agent which is deposited in the throat of the user is less than 15% of the active agent in the metered dose.

9. A drug delivery system as claimed in claim 1, wherein the powder composition further comprises an additive which is a force control agent.

10. A drug delivery system as claimed in claim 1, wherein the powder composition further comprises carrier particles comprising a physiologically acceptable and inert excipient material.

11. A drug delivery system as claimed in claim 10, wherein the carrier particles are coarse, fine or a combination of coarse and fine particles.

12. A drug delivery system as claimed in claim 10, wherein the composition comprises fine carrier particles which have a force control agent present on their surfaces.

13. A drug delivery system as claimed in claim 1, wherein the active particles have a force control agent present on their surfaces.

14. A drug delivery system as claimed in claim 1, wherein the powder composition has a powder density of at least 0.3 g/cc.

15. A method of preparing a dry powder composition for inclusion in a drug delivery system as claimed in claim 1, wherein the composition comprises an additive material which is a force control agent and wherein the majority of the additive material is present on the surface of particles of other material.

16. A method as claimed in claim 15, wherein the particles of other material include particles of a pharmaceutically active agent and wherein the additive is present on the surfaces of particles of the active agent.

17. A method as claimed in claim 16, wherein the method includes the preparation of composite particles comprising the active agent and the additive material.

18. A method as claimed in claim 15, wherein the particles of other material include carrier particles comprising a physiologically acceptable and inert excipient material and wherein the additive is present on the surfaces of the cattier particles.

19. A method as claimed in claim 18, wherein the method includes the preparation of composite particles comprising the physiologically acceptable and inert excipient material and the additive material.

20. A method as claimed in claim 17, wherein the composite particles are prepared by co-spray drying the active and additive materials.

21. A method as claimed in claim 20, wherein the spray drying involves the formation of droplets moving at a controlled velocity.

22. A method as claimed in claim 21, wherein the droplets are formed using an ultrasonic nebuliser, electrospray nozzles or vibrating orifice nozzles.

23. A method as claimed in claim 17, wherein the composite particles are prepared by co-milling particles of the active material and particles of the additive material.

24. A method as claimed in claim 23, wherein the particles of active material are formed by a milling step prior to their co-milling with the additive material.

25. A method as claimed in claim 24, wherein the particles of active material are jet-milled prior to their co-milling with the additive material.

26. A method as claimed in claim 23, wherein the active and additive particles are co-jet milled at pressures from about 0.1 to about 3 bar.

27. A method as claimed in claim 23, wherein the composite particles subsequently undergo a compressive process, wherein the particles are compressed within a gap of predetermined width.

28. A method as claimed in claim 19, wherein the composite particles are prepared by co-milling particles of the excipient material and particles of the additive material.

29. A method as claimed in claim 28, wherein the particles of excipient material have a median diameter of about 3 to about 40 μm.

30. A method as claimed in claim 28, wherein the active and additive particles are co-jet milled at pressures from about 0.1 to about 3 bar.
